(19) United States
(12) Patent Application Publication
(10) Pub. No.: US 2007/0149442 A1
(43) Pub. Date: Jun. 28, 2007

(54) Non-hygroscopic compositions of enterostatin

(76) Inventor: Byron Rubin, Honeoye Falls, NY (US)

Correspondence Address: JONES DAY, 222 EAST 41ST ST, NEW YORK, NY, 10017, US

(21) Appl. No.: 11/638,046

(22) Filed: Dec. 12, 2006

Related U.S. Application Data
(60) Provisional application No. 60/750,208, filed on Dec. 13, 2005.

(51) Int. Cl. A61K 38/12

(52) U.S. Cl. 514/9

(57) ABSTRACT

The present invention provides pharmaceutical compositions of enterostatin that can display advantageous hygroscopicity, advantageous stability, or both. The pharmaceutical compositions of enterostatin can be useful for the manufacture of an pharmaceutical product comprising enterostatin.

This application claims the benefit of priority of U.S. provisional application No. 60/750,208, filed Dec. 13, 2005, the contents of which are hereby incorporated by reference in their entireties.

### 1. FIELD OF THE INVENTION

The present invention provides novel non-hygroscopic pharmaceutical compositions or formulations of peptides that modulate F₁-ATPase activity. The invention further provides the use of the novel non-hygroscopic compositions or formulations, for example, in the treatment of conditions related to enterostatin activity or F₁-ATPase activity, such as obesity and diabetes. The non-hygroscopic compositions and formulations of the invention can be used for the treatment or prevention of conditions related to enterostatin activity or F₁-ATPase activity, such as obesity and diabetes, with little or no concern for degradation or instability of the active peptide.

### 2. BACKGROUND OF THE INVENTION

Obesity is a complex condition that is increasingly affecting the population worldwide. According to the World Health Organization, in 1995 there were an estimated 200 million obese adults worldwide and another 18 million under-five children classified as overweight. As of 2000, the number of obese adults had increased to over 300 million. See Formiguera et al., 2004, Best Practice & Research Clinical Gastroenterology, 18:6, 1125-1146.

Overweight or obesity has been shown to increase risk for several diseases and health conditions, including hypertension, dyslipidemia (high total cholesterol or high levels of triglycerides), type II diabetes, coronary heart disease, stroke, gallbladder disease, osteoarthritis, sleep apnea and respiratory problems and some cancers (for example, endometrial, breast, and colon). See, e.g., U.S. National Center for Chronic Disease Prevention and Health Promotion. Its health consequences range from increased risk of premature death to serious chronic conditions that reduce the overall quality of life.

Various therapies have been proposed or tested for the modulation of physiological processes that might lead to conditions such as overweight or obesity. See Orzano et al., 2004, J. Am. Board Fam. Pract. 17(5):359-69. One of these is enterostatin.

Enterostatin is a peptide that has shown promise in modulating dietary fat preference in rodents. See, e.g., Erlanson-Albertsson et al., 1991, Physiol. Behav. 49:1191-1194; Okada et al., 1991, Physiol. Behav. 49:1185-1189; Shargill et al., 1991, Brain Res. 544:137-140. Enterostatin is generated by tryptic activation of procolipase in the intestine or stomach to generate colipase. Colipase binds and activates the enzyme lipase to metabolize fats in the intestine. The propeptide enterostatin is believed to reduce dietary fat preference in mammals as demonstrated in rodent studies. See, Erlanson-Albertsson et al., 1991, Okada et al., 1991, Physiol. Behav. 49:1185-1189, Shargill et al., 1991. Accordingly, studies of decreasing appetite in mammals by administering an effective amount of an enterostatin peptide have been reported. See, Erlanson-Albertsson, 1996, U.S. Pat. No. 5,494,894. Human studies concerning endogenous enterostatin have been reported. See e.g., Prasad et al., 1999, J. Clin. Endocrinol. Metab. 84:937-941; Kovacs et al., 2003, British J. Nutrition 90:207-214.

In developing novel methods of administering enterostatin, it was discovered that conventional forms of enterostatin can take on too much water in ambient or storage conditions for the efficient manufacture of an enterostatin pharmaceutical product. Conventional forms of enterostatin that absorb too much water can degrade over time and can be difficult to measure and administer reproducibly. Those of skill in the art will recognize that the hygroscopicity of conventional forms of enterostatin can be too great for efficient storage and use in conventional pharmaceutical tablets or capsules.

Stable compositions or formulations of enterostatin are needed for the pharmaceutical use of such peptides for the modulation of food intake and the treatment or prevention of conditions associated with enterostatin or F₁-ATPase activity, such as obesity or diabetes.

### 3. SUMMARY OF THE INVENTION

The present invention provides novel, non-hygroscopic pharmaceutical compositions comprising peptides such as enterostatin. The non-hygroscopic pharmaceutical compositions of the invention can display increased stability in ambient conditions or in storage conditions. Accordingly, the novel, non-hygroscopic pharmaceutical compositions of the invention are useful in and for the manufacture of stable pharmaceutical products for storage and use. The present invention thus provides pharmaceutical compositions and formulations comprising a peptide with enterostatin or F₁-ATPase activity that have increased shelf life, that have reduced moisture content and/or that absorb less moisture in storage, ambient or high humidity conditions.

The novel, non-hygroscopic pharmaceutical compositions can be used for the treatment or prevention of any condition or disorder for which the peptide itself is useful. For instance, the novel, non-hygroscopic pharmaceutical compositions of the invention can be used for the treatment or prevention of conditions related to enterostatin or F₁-ATPase activity, such as described in U.S. provisional application No. 60/750,206, filed Dec. 13, 2005, entitled “Methods of Treating Obesity Using Enterostatin,” the contents of which are hereby incorporated by reference in its entirety. Exemplary disorders or conditions related to enterostatin or F₁-ATPase activity include, but are not limited to, overweight, obesity, metabolic disorders, hypertension, lipid related disorders, and type II diabetes.

In one aspect, the present invention provides a non-hygroscopic pharmaceutical composition comprising an enterostatin peptide and one or more non-hygroscopic additives. In certain embodiments, the composition comprises a formulation that reduces or eliminates contact of the active peptide with moisture. Exemplary non-hygroscopic additives include dibasic calcium phosphate anhydrous, calcium sulfate, calcium silicate, powdered cellulose, dextrose, lactitol, mannitol or mixtures thereof. Exemplary compositions, methods of their preparation and methods of their use are described in the sections below.

In another aspect, the present invention provides a non-hygroscopic pharmaceutical composition comprising an enterostatin peptide encapsulated by a non-hygroscopic matrix. Suitable encapsulating matrices include gelatins, such as type A gelatins and type B gelatins, celluloses, such as hydroxypropyl methylcellulose, starches and gum acacia. Exemplary encapsulated compositions, methods of their preparation and methods of their use are described in the sections below.

In a further aspect, the present invention provides a non-hygroscopic pharmaceutical composition comprising a non-hygroscopic solid dispersion of an enterostatin peptide. Suitable solid dispersions include those that comprise a matrix forming agent, one or more optional fillers and the enterostatin peptide. An exemplary matrix forming agent can be selected from the group consisting of hydroxyethylcellulose, hydroxypropyl cellulose (HPC), hydroxypropyl methylcellulose (HPMC), HPMC phthalate, polyvinyl pyrrolidone (PVP), polyethylene glycol (PEG), polyglycolized glycerides, cyclodextrins and carbomers. The enterostatin peptide is dispersed or dissolved in the matrix and optional filler(s).

In aspects of the invention, the enterostatin peptide can be any peptide with enterostatin or F₁-ATPase activity. In particular embodiments, the enterostatin peptide has a sequence selected from the group consisting of consisting of APGPR (SEQ ID NO:1), VPDPR (SEQ ID NO:2) and VPGPR (SEQ ID NO:3).

In another aspect, the present invention provides methods of treating or preventing a metabolic condition or disorder with a pharmaceutical composition of the invention. In certain embodiments, the condition or disorder is associated with F₁-ATPase activity or enterostatin activity. In particular embodiments, the condition is associated with enterostatin deficiency. The methods comprise the step of administering an effective amount of a pharmaceutical composition or formulation of the invention to a subject in need thereof. The methods are useful for the treatment or prevention of any condition associated with enterostatin including, but not limited to, overweight or obesity.

### 4. DETAILED DESCRIPTION OF THE INVENTION

4.1 Definitions

When referring to the compositions and formulations of the invention, the following terms have the following meanings unless indicated otherwise.

The term “enterostatin” encompasses the propeptide of procolipase, as is known to those of skill in the art. Exemplary enterostatins have an amino acid sequence selected from the group consisting of APGPR (SEQ ID NO:1), VPDPR (SEQ ID NO:2) and VPGPR (SEQ ID NO:3). In a preferred embodiment, the enterostatin has an amino acid sequence of APGPR (SEQ ID NO:1).

“Hygroscopic” refers to a substance that is capable of readily absorbing moisture from, for example, the atmosphere as understood by those of skill in the art. In certain embodiments, “hygroscopicity” refers to sorption, implying an acquired amount or state of water sufficient to affect the physical or chemical properties of the substance (Eds. J. Swarbrick and J. C. Boylan, Encyclopedia of Pharmaceutical Technology, Vol. 10, p. 33).

A “non-hygroscopic” composition refers to a composition that does not readily absorb moisture from an atmosphere, for instance a humid atmosphere, under conditions recognized by those of skill in the art. In certain embodiments, the non-hygroscopic composition reduces or eliminates contact between moisture and the active ingredient of the composition. In certain embodiments, a non-hygroscopic composition absorbs less than 20% moisture, by weight, at about 50% relative humidity. In certain embodiments, a non-hygroscopic composition absorbs less than 15% moisture, by weight, at about 50% relative humidity. In certain embodiments, a non-hygroscopic composition absorbs less than 10% moisture, by weight, at about 50% relative humidity. In certain embodiments, a non-hygroscopic composition absorbs less than 5% moisture, by weight, at about 50% relative humidity. In certain embodiments, a non-hygroscopic composition absorbs less than 4% moisture, by weight, at about 50% relative humidity. In certain embodiments, a non-hygroscopic composition absorbs less than 3% moisture, by weight, at about 50% relative humidity. In certain embodiments, a non-hygroscopic composition absorbs less than 20% moisture, by weight, at about 50% relative humidity. Moisture absorbance can be measured under conditions known to those of skill in the art for a length of time known to those of skill in the art. In certain embodiments, moisture absorbance can be measured under heat for accelerated storage conditions known to those of skill. In certain embodiments, moisture absorbance can be measured for at least 1, 2, 3, 4, 5, 10, 15, 20, 25, 30 days or 1, 2, 3, 4, 5 or 6 months.

The term “unbound water” as used herein refers to water that is not present in the form of a stable solvate or hydrate of one or more components of a pharmaceutical composition.

The term “substantially free of unbound water” typically means that less than about 5 weight percent, preferably less than about 1 weight percent, and more preferably, less than about 0.1 weight percent, of water is present.

“Pharmaceutically acceptable salt” refers to any salt of a compound of this invention which retains its biological properties and which is not toxic or otherwise undesirable for pharmaceutical use. Such salts may be derived from a variety of organic and inorganic counter-ions well known in the art and include. Such salts include: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) salts formed when an acidic proton present in the parent compound either (a) is replaced by a metal ion, e.g., an alkali metal ion, an alkaline earth ion or an aluminum ion, or alkali metal or alkaline earth metal hydroxides, such as sodium, potassium, calcium, magnesium, aluminum, lithium, zinc, and barium hydroxide, ammonia or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Salts further include, by way of example only, sodium, potassium, calcium, magnesium, ammonium, tetraalkylammonium and the like, and when the compound contains a basic functionality, salts of non-toxic organic or inorganic acids, such as hydrohalides, e.g. hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate and the like.

The term “physiologically acceptable cation” refers to a non-toxic, physiologically acceptable cationic counterion of an acidic functional group. Such cations are exemplified by sodium, potassium, calcium, magnesium, ammonium and tetraalkylammonium cations and the like.

“Solvate” refers to a compound of the present invention or a salt thereof, that further includes a stoichiometric or non-stoichiometric amount of solvent bound by non-covalent intermolecular forces. Where the solvent is water, the solvate is a hydrate.

It is to be understood that compounds having the same molecular formula but differing in the nature or sequence of bonding of their atoms or in the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, when it is bonded to four different groups, a pair of enantiomers is possible. An enantiomer can be characterized by the absolute configuration of its asymmetric center and is designated (R) or (S) according to the rules of Cahn and Prelog (Cahn et al., 1966, Angew. Chem. 78:413-447, Angew. Chem., Int. Ed. Engl. 5:385-414 (errata: Angew. Chem., Int. Ed. Engl. 5:511); Prelog and Helmchen, 1982, Angew. Chem. 94:614-631, Angew. Chem. Internat. Ed. Eng. 21:567-583; Mata and Lobo, 1993, Tetrahedron: Asymmetry 4:657-668) or can be characterized by the manner in which the molecule rotates the plane of polarized light and is designated dextrorotatory or levorotatory (i.e., as (+)- or (−)-isomers, respectively). A chiral compound can exist as either individual enantiomer or as a mixture thereof. A mixture containing equal proportions of enantiomers is called a “racemic mixture”.

In certain embodiments, the compounds of this invention may possess one or more asymmetric centers; such compounds can therefore be produced as the individual (R)- or (S′)-enantiomer or as a mixture thereof. Unless indicated otherwise, for example by designation of stereochemistry at any position of a formula, the description or naming of a particular compound in the specification and claims is intended to include both individual enantiomers and mixtures, racemic or otherwise, thereof. Methods for determination of stereochemistry and separation of stereoisomers are well-known in the art. In particular embodiments, the present invention provides the stereoisomers of the compounds depicted herein upon treatment with base.

In certain embodiments, the compounds or compositions of the invention are “stereochemically pure.” A stereochemically pure compound or composition has a level of stereochemical purity that would be recognized as “pure” by those of skill in the art. Of course, this level of purity will be less than 100%. In certain embodiments, “stereochemically pure” designates a compound or composition that is substantially free of alternate isomers. In particular embodiments, the compound or composition is 85%, 90%, 91%, 92%, 93%, 94%, 95%, 96%, 97%, 98%, 99%, 99.5% or 99.9% free of other isomers.

The amino acid notations used herein for the twenty genetically encoded L-amino acids are conventional and are as follows:

| | One-Letter | Three Letter |
|---|---|---|
| Amino Acid | Abbreviation | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

As used herein, unless specifically delineated otherwise, the three-letter amino acid abbreviations designate amino acids in the L-configuration. Amino acids in the D-configuration are preceded with a “D-.” For example, Arg designates L-arginine and D-Arg designates D-arginine. Likewise, the capital one-letter abbreviations refer to amino acids in the L-configuration. Lower-case one-letter abbreviations designate amino acids in the D-configuration. For example, “R” designates L-arginine and “r” designates D-arginine.

Unless noted otherwise, when peptide or polypeptide sequences are presented as a series of one-letter and/or three-letter abbreviations, the sequences are presented in the N-terminal to C-terminal direction, in accordance with common practice.

In preferred embodiments, any peptide or amino acid of the invention is in the L form, unless otherwise indicated.

The term “subject” refers to an animal such as a mammal, including, but not limited to, primate (e.g., human), cow, sheep, goat, horse, dog, cat, rabbit, rat, mouse and the like. In preferred embodiments, the subject is a human.

“Therapeutically effective amount” means an amount of a compound or complex or composition that, when administered to a subject for treating a disease, is sufficient to effect such treatment for the disease. A “therapeutically effective amount” can vary depending on, inter alia, the compound, the disease and its severity, and the age, weight, etc., of the subject to be treated.

“Treating” or “treatment” of any disease or disorder refers, in one embodiment, to ameliorating the disease or disorder (i.e., arresting or reducing the development of the disease or at least one of the clinical symptoms thereof) that exists in a subject. In another embodiment, “treating” or “treatment” refers to ameliorating at least one physical parameter, which may be indiscernible by the subject. In yet another embodiment, “treating” or “treatment” refers to modulating the disease or disorder, either physically (e.g., stabilization of a discernible symptom) or physiologically (e.g., stabilization of a physical parameter) or both. In yet another embodiment, “treating” or “treatment” refers to delaying the onset of the disease or disorder.

### 4.2 Embodiments of the Invention

The present invention provides novel, non-hygroscopic pharmaceutical compositions or formulations comprising peptides having F₁-ATPase activity or enterostatin activity. The non-hygroscopic pharmaceutical compositions or formulations can display advantageous hygroscopicity and/or advantageous stability. The non-hygroscopic pharmaceutical compositions or formulations are useful, for example, as pharmaceutical products, for the manufacture of pharmaceutical products and for long term storage of the peptides. In particular embodiments, the non-hygroscopic pharmaceutical compositions or formulations are useful in oral dosage forms including, but not limited to, tablets, capsules cachets, dragees and the like that do not necessarily use or are not necessarily made under anhydrous conditions, for instance, those made under conditions with some moisture.

The non-hygroscopic pharmaceutical composition has a level of hygroscopicity considered low by those of skill in the art. For instance, the non-hygroscopic pharmaceutical composition can have a hygroscopicity considered acceptable to those of skill in the art for the manufacture, storage and convenient use of a pharmaceutical. In certain embodiments, a non-hygroscopic pharmaceutical composition of the invention will absorb less than 10%, 9%, 8%, 7%, 6%, 5%, 4%, 3%, 2% or 1% water by weight in an atmosphere of normal humidity. In certain embodiments, a non-hygroscopic pharmaceutical composition of the invention will remain solid for at least 1, 2, 3, 4, 5, 10, 15 or 20 days at least 25%, 50% or 75% humidity. In preferred embodiments, a non-hygroscopic pharmaceutical composition of the invention will remain solid for at least 4 or 10 days at least 58% humidity. In certain embodiments, the non-hygroscopic pharmaceutical composition will gain less than 35%, 30%, 25% or 20% water, by weight, when moved from 5% to 95% relative humidity under techniques known to those of skill in the art. In certain embodiments, the non-hygroscopic pharmaceutical composition will lose less than 35%, 30%, 25% or 20% water, by weight, when moved from 95% to 5% relative humidity under techniques known to those of skill in the art. In certain embodiments, the non-hygroscopic pharmaceutical composition will gain less than 35%, 30%, 25% or 20% water, by weight, when moved from 5% to 95% relative humidity, and they will lose less than 35%, 30%, 25% or 20% water, by weight, when moved from 95% to 5% relative humidity.

Hygroscopicity of a composition or formulation of the invention can be measured under conditions apparent to those of skill in the art. For instance, in certain embodiments, hygroscopicity is measured under ambient or storage conditions. In certain embodiments, hygroscopicity is measured under accelerated storage conditions, for instance under heat.

In certain embodiments, the non-hygroscopic pharmaceutical composition of the invention comprises an enterostatin peptide and a non-hygroscopic additive. The non-hygroscopic additive can be any pharmaceutically compatible non-hygroscopic additive known to those of skill in the art. In particular embodiments, the non-hygroscopic additive is selected from the group consisting of dibasic calcium phosphate anhydrous, calcium sulfate, calcium silicate, powdered cellulose, dextrose, lactitol, mannitol and mixtures thereof. Exemplary compositions or formulations, methods of their preparation and methods of their use are described in the sections below.

In further embodiments, the present invention provides a non-hygroscopic pharmaceutical composition comprising an enterostatin peptide encapsulated by a non-hygroscopic matrix. The non-hygroscopic matrix can be any non-hygroscopic matrix known to those of skill in the art. In particular embodiments, the non-hygroscopic matrix is selected from the group consisting of gelatins, such as type A gelatins and type B gelatins, celluloses, such as hydroxypropyl methylcellulose, starches and gum acacia. The composition can further comprise one or more pharmaceutically acceptable carriers, diluents or excipients known to those of skill in the art. Exemplary encapsulated compositions or formulations, methods of their preparation and methods of their use are described in the sections below.

In further embodiments, the present invention provides a non-hygroscopic pharmaceutical composition comprising a non-hygroscopic solid dispersion of an enterostatin peptide. Suitable solid dispersions include those that comprise a matrix forming agent, one or more optional fillers and the enterostatin peptide. The matrix forming agent can be any matrix forming agent capable of forming a solid dispersion known to those of skill in the art. In certain embodiments, the matrix forming agent can be selected from the group consisting of hydroxyethylcellulose, hydroxypropylcellulose (HPC), hydroxypropyl methylcellulose (HPMC), HPMC phthalate, polyvinyl pyrrolidone (PVP), polyethylene glycol (PEG), polyglycolized glycerides, cyclodextrins and carbomers. The composition can further comprise one or more pharmaceutically acceptable carriers, diluents or excipients known to those of skill in the art.

In the non-hygroscopic pharmaceutical compositions or formulations, the components can be in neutral forms, one component can be in a salt form, or more than one component can be in a salt form. Exemplary salt forms are described in detail in the sections below.

In certain embodiments, the non-hygroscopic pharmaceutical compositions or formulations of the invention comprise a crystalline form of enterostatin. Crystalline forms of the invention have one or more crystalline property that would be recognized by those of skill in the art. For instance, crystalline forms of the invention can have one or more properties selected from the group consisting of birefringence, defined X-ray powder diffraction peaks, defined X-ray diffraction peaks or spots, defined melting temperature, defined shape, or any other crystalline property known to those of skill in the art. In certain embodiments, the present invention provides crystalline forms of enterostatin peptides.

As the non-hygroscopic pharmaceutical compositions or formulations of the invention find use, for example, in and for the manufacture of pharmaceutical products, the present invention also encompasses solvates of the non-hygroscopic pharmaceutical compositions or formulations of the invention. As will be recognized by those of skill in the art, a solvate of a composition or formulation of the invention comprises the non-hygroscopic pharmaceutical compositions or formulations coordinated with one or more solvent molecules. In preferred embodiments, the solvent is pharmaceutically acceptable. In particularly preferred embodiments, the solvent is water, i.e. the solvate is a hydrate.

4.3 Enterostatin Peptides of the Invention

The enterostatin non-hygroscopic pharmaceutical compositions or formulations comprise an enterostatin peptide. The enterostatin peptide can be any enterostatin peptide known to those of skill in the art. The enterostatin peptide can be from the same species as a subject to be treated, or the enterostatin can be from a different species. In preferred embodiments, the enterostatin peptide is from the same species as the subject. Exemplary enterostatin peptides include human, rat, mouse, porcine, canine and equine enterostatin peptides. Methods of making enterostatin peptides of the invention are discussed in a section below.

In certain embodiments, the enterostatin peptide is a full-length enterostatin peptide. Exemplary enterostatin peptides have an amino acid sequence selected from the group consisting of APGPR (SEQ ID NO:1), VPDPR (SEQ ID NO:2) and VPGPR (SEQ ID NO:3). The enterostatin compositions or formulations of the invention can comprise a single enterostatin, or they can comprise multiple enterostatins. Preferred is APGPR (SEQ ID NO:1). Methods of making the enterostatin peptides are described in detail below.

In preferred embodiments, the enterostatin is substantially pure. In this context the term “substantially pure” indicates that the enterostatin is substantially free of contaminants not intended to be administered. Examples include peptide or amino acid contaminants and peptide synthesis reagents. In certain embodiments, the enterostatin is 85%, 90%, 91%, 92%, 93%, 94%, 95%, 96%, 97%, 98%, 99%, 99.5% or 99.9% pure. As discussed in detail the section below, enterostatin can be formulated for administration with one or more carriers, excipients or diluents.

The enterostatin can comprise free termini or blocked termini according to the judgment of those of skill in the art. Useful blocked termini include a C-terminal amide or an N-terminal acetyl, or both. In preferred embodiments, the enterostatin has free N- and C-termini.

The enterostatin peptide can be in a neutral form, or in a salt form. The salt form can be any salt form known to those of skill in the art. Particularly useful salt forms are those that are coordinated with acetate, chloride, sulfate and phosphate. Acetate and chloride salts are preferred.

Where a compound of the present invention, e.g. an enterostatin peptide, is substituted with a basic moiety, an acid addition salt can be formed. The acid which can be used to prepare an acid addition salt includes preferably that which produces, when combined with the free base, a pharmaceutically acceptable salt, that is, a salt whose anion is non-toxic to a patient in the pharmaceutical doses of the salt. Pharmaceutically acceptable salts within the scope of the invention are those derived from the following acids: mineral acids such as hyrochloric acid, hydrobromic acid, sulfuric acid, phosphoric acid, sulfamic acid and nitric acid; and organic acids such as acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids.

The corresponding acid addition salts include hydrohalides, e.g. hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate and the like.

According to a further feature of the invention, acid addition salts of the compounds of this invention can be prepared by reaction of the free base with the appropriate acid, by the application or adaptation of known methods. For example, the acid addition salts of the compounds of this invention can be prepared either by dissolving the free base in aqueous or aqueous-alcohol solution or other suitable solvents containing the appropriate acid and isolating the salt by evaporating the solution, or by reacting the free base and acid in an organic solvent, in which case the salt separates directly or can be obtained by concentration of the solution.

The acid addition salts of the compounds of this invention, e.g. enterostatin peptides, can be regenerated from the salts by the application or adaptation of known methods. For example, parent compounds of the invention can be regenerated from their acid addition salts by treatment with an alkali, e.g., aqueous sodium bicarbonate solution or aqueous ammonia solution.

Where a compound of the invention, e.g. enterostatin peptides, is substituted with an acid moiety, base addition salts can be formed. Pharmaceutically acceptable salts, including for example alkali and alkaline earth metal salts, within the scope of the invention are those derived from the following bases: sodium hydride, sodium hydroxide, potassium hydroxide, calcium hydroxide, magnesium hydroxide, aluminum hydroxide, lithium hydroxide, zinc hydroxide, barium hydroxide, and organic amines such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Metal salts of compounds of the present invention, e.g. enterostatin peptides, can be obtained by contacting a hydride, hydroxide, carbonate or similar reactive compound of the chosen metal in an aqueous or organic solvent with the free acid form of the compound. The aqueous solvent employed may be water or it may be a mixture of water with an organic solvent, preferably an alcohol such as methanol or ethanol, a ketone such as acetone, an aliphatic ether such as tetrahydrofuran, or an ester such as ethyl acetate. Such reactions are normally conducted at ambient temperature but they may, if desired, be conducted with heating.

Amine salts of compounds of the present invention, e.g. enterostatin peptides, can be obtained by contacting an amine in an aqueous or organic solvent with the free acid form of the compound. Suitable aqueous solvents include water and mixtures of water with alcohols such as methanol or ethanol, ethers such as tetrahydrofuran, nitrites, such as acetonitrile, or ketones such as acetone. Amino acid salts may be similarly prepared.

The base addition salts of the compounds of this invention, e.g. enterostatin peptides, can be regenerated from the salts by the application or adaptation of known methods. For example, parent compounds of the invention can be regenerated from their base addition salts by treatment with an acid, e.g., hydrochloric acid.

As well as being useful in themselves as active compounds, salts of compounds of the invention, e.g. enterostatin peptides, are useful for the purposes of purification of the compounds, for example by exploitation of the solubility differences between the salts and the parent compounds, side products and/or starting materials by techniques well known to those skilled in the art.

In certain embodiments, the enterostatin peptide is in the form of a co-complex with a guest molecule. In particular embodiments, the guest molecule is a guest molecule that reduces the hygroscopicity of the enterostatin peptide. Such enterostatin co-complexes are fully described in U.S. provisional application No. 60/750,207, filed Dec. 13, 2005, entitled “Stable Solid Forms of Enterostatin,” the contents of which are hereby incorporated by reference in their entirety.

4.4 Pharmaceutical Compositions of the Invention Comprising a Non-Hygroscopic Additive

In certain embodiments, the non-hygroscopic pharmaceutical composition of the invention comprises an enterostatin peptide and a non-hygroscopic additive or a system that reduces or prevents contact of the peptide with moisture. In certain embodiments, the amount of the non-hygroscopic additive is sufficient to produce non-hygroscopic compositions.

The non-hygroscopic pharmaceutical composition comprises an enterostatin, or a salt or co-complex thereof, as described in the sections above. Although the preferred salt is enterostatin acetate or enterostatin chloride, any other salt or derivative of enterostatin that is suitable for oral administration, or mixtures of enterostatin salts and derivatives can be used. The non-hygroscopic additive can be any non-hygroscopic additive known to those of skill in the art. Exemplary non-hygroscopic additives are described in detail below.

In certain embodiments, the non-hygroscopic additive is sufficient to yield a non-hygroscopic pharmaceutical composition of the invention that absorbs less than 10%, 9%, 8%, 7%, 6%, 5%, 4%, 3%, 2% or 1% water by weight in an atmosphere of normal humidity. In certain embodiments, the non-hygroscopic additive is sufficient to yield a non-hygroscopic pharmaceutical composition of the invention that will remain solid for at least 1, 2, 3, 4, 5, 10, 15 or 20 days at least 25%, 50% or 75% humidity. In preferred embodiments, the non-hygroscopic additive is sufficient to yield a non-hygroscopic pharmaceutical composition of the invention that will remain solid for at least 4 or 10 days at at least 58% humidity. In certain embodiments, the non-hygroscopic additive is sufficient to yield a non-hygroscopic pharmaceutical composition of the invention that will gain less than 35%, 30%, 25% or 20% water, by weight, when moved from 5% to 95% relative humidity under techniques known to those of skill in the art. In certain embodiments, the non-hygroscopic additive is sufficient to yield a non-hygroscopic pharmaceutical composition of the invention that will lose less than 35%, 30%, 25% or 20% water, by weight, when moved from 95% to 5% relative humidity under techniques known to those of skill in the art. In certain embodiments, the non-hygroscopic additive is sufficient to yield a non-hygroscopic pharmaceutical composition of the invention that will gain less than 35%, 30%, 25% or 20% water, by weight, when moved from 5% to 95% relative humidity, and that will lose less than 35%, 30%, 25% or 20% water, by weight, when moved from 95% to 5% relative humidity.

In certain embodiments, the compositions of the present invention may be prepared as solid dosage forms such as bulk powders, tablets, caplets, pellets, capsules, sachets, granules, and any other dosage form suitable for oral administration.

The non-hygroscopic additive is used in the present invention to enhance the non-hygroscopic properties of the composition. The non-hygroscopic additive can be any material which assists in reducing moisture absorption of the enterostatin and/or retains the non-hygroscopic properties of the composition.

The ratio of enterostatin to non-hygroscopic additive can be any ratio that yields a non-hygroscopic composition. In certain embodiments, the ratio is within the range of 10:1 to 1:10, 5:1 to 1:5, 4:1 to 1:4, or 2:1 to 1:1 additive to enterostatin, or the range is about 1:1. In particular embodiments the ratio is about 10:1, 5:1, 4:1, 3:1, 2.5:1, 2:1, 1:1, 1:2.5, 1:3, 1:4, 1:5, or 1:10 additive to enterostatin.

Preferred non-hygroscopic additives include dibasic calcium phosphate anhydrous, calcium sulfate, calcium silicate, powdered cellulose, dextrose, lactitol, mannitol and mixtures thereof. The non-hygroscopic additive can be obtained from any source known to those of skill in the art.

The non-hygroscopic additive can be present in any amount in the composition that is sufficient to yield a non-hygroscopic composition. In certain embodiments, the non-hygroscopic additive is present in an amount of from about 1% to about 90% of the weight of the final composition, of from about 10% to about 90% of the weight of the final composition, of from about 20% to about 90% of the weight of the final composition, of from about 25% to about 90% of the weight of the final composition or of from about 50% to about 90% of the weight of the final composition. In particular embodiments, the non-hygroscopic additive is present in an amount of about 10%, 15%, 20%, 25%, 30%, 33%, 40%, 50%, 60%, 67%, 70%, 75%, 80%, 85% or 90% of the final composition.

The pharmaceutical compositions of the invention optionally comprise at least one additional excipient. The additional excipients include, for instance, pharmaceutical lubricants, binders, disintegrators, glidants, adsorbents, and mixtures thereof. Such “other ingredients” are described in the sections below.

It should be noted that non-hygroscopic pharmaceutical compositions of the present invention may nevertheless include some hygroscopic ingredients; however, the overall composition must be substantially non-hygroscopic. Further, suitable excipients for use in such non-hygroscopic pharmaceutical compositions include hydrated excipients, such as α-lactose monohydrate and the like.

4.5 Pharmaceutical Compositions of the Invention Encapsulated by Non-Hygroscopic Shells

In further embodiments aspect, the present invention provides a non-hygroscopic pharmaceutical composition comprising an enterostatin peptide encapsulated by a non-hygroscopic matrix.

In certain embodiments, the non-hygroscopic matrix is sufficient to yield a non-hygroscopic pharmaceutical composition of the invention that absorbs less than 10%, 9%, 8%, 7%, 6%, 5%, 4%, 3%, 2% or 1% water by weight in an atmosphere of normal humidity. In certain embodiments, the non-hygroscopic matrix is sufficient to yield a non-hygroscopic pharmaceutical composition of the invention that will remain solid for at least 1, 2, 3, 4, 5, 10, 15 or 20 days at least 25%, 50% or 75% humidity. In preferred embodiments, the non-hygroscopic matrix is sufficient to yield a non-hygroscopic pharmaceutical composition of the invention that will remain solid for at least 4 or 10 days at at least 58% humidity. In certain embodiments, the non-hygroscopic matrix is sufficient to yield a non-hygroscopic pharmaceutical composition of the invention that will gain less than 35%, 30%, 25% or 20% water, by weight, when moved from 5% to 95% relative humidity under techniques known to those of skill in the art. In certain embodiments, the non-hygroscopic matrix is sufficient to yield a non-hygroscopic pharmaceutical composition of the invention that will lose less than 35%, 30%, 25% or 20% water, by weight, when moved from 95% to 5% relative humidity under techniques known to those of skill in the art. In certain embodiments, the non-hygroscopic matrix is sufficient to yield a non-hygroscopic pharmaceutical composition of the invention that will gain less than 35%, 30%, 25% or 20% water, by weight, when moved from 5% to 95% relative humidity, and that will lose less than 35%, 30%, 25% or 20% water, by weight, when moved from 95% to 5% relative humidity.

The non-hygroscopic matrix can be any non-hygroscopic matrix known to those of skill in the art. In particular embodiments, the non-hygroscopic matrix is selected from the group consisting of gelatins, such as type A gelatins and type B gelatins, celluloses, such as hydroxypropyl methylcellulose, starches and gum acacia. The composition can further comprise one or more pharmaceutically acceptable carriers, diluents or excipients known to those of skill in the art. Exemplary encapsulated compositions, methods of their preparation and methods of their use are described in the sections below.

In certain embodiments, the matrix forming material can be used for the formation of a shell around the enterostatin peptide. In certain embodiments, the matrix forming material can be formulated with a plasticizing agent

The shell may be either a hard or soft capsule shell, and may comprise a matrix forming material and a plasticizing agent. A wide variety of matrix forming materials are suitable for use in non-hygroscopic pharmaceutical compositions of the present invention, and the selection of specific materials may be based, at least in part, on factors such as the specific results to be achieved. Examples of specific materials include without limitation, gelatins, such as type A gelatins and type B gelatins, celluloses, such as hydroxypropyl methylcellulose, starches and gum acacia. Other specific matrix forming materials that may be particularly desired in view of a given overall dosage form can be determined by those of ordinary skill in the art.

The specific amount of matrix forming material used in the shell formulation may be determined in part by a variety of factors, including the type of shell to be formed (i.e. hard or soft), and by the amount and type of other constituents or additives that are to be included in the shell. However, in one aspect, the amount of matrix forming material may be from about 20% w/w to about 70% w/w of the shell. In another aspect, the amount may be from about 30% w/w to about 50% w/w of the shell.

Many plasticizing agents are known, and may also be used in the shell of the present dosage form. One basis for selecting a particular plasticizing agent may be the solubility of that agent in the fill material to be used in the composition. In this context, the fill material is the portion of the pharmaceutical composition within the shsell. In one aspect, the plasticizing agent may have a solubility of less than about 10% w/w in the fill material. In another aspect, the solubility of the plasticizing agent in the fill material may be less than about 5% w/w. In yet another aspect, the solubility may be less than about 1% w/w. In a further aspect, the solubility of the plasticizing agent may be less than about 0.5% w/w. Lowered solubility in the fill material substantially impedes the migration of the plasticizing agent out of the shell and into the fill material. Examples of specific plasticizing agents displaying such limited solubilities in many hydrophilic surfactant materials include without limitation: sorbitol, sorbitanes, xylitol, maltitol, maltitol syrup, partially dehydrated hydrogenated glucose syrups, hydrogenated starch hydrolysate, polyhydric alcohols having an equilibrium relative humidity of greater than or equal to 80%, carrageenan, polyglycerol, non-crystallizing solutions of sorbitol, glucose, fructose, glucose syrups, and mixtures and equivalents thereof.

In certain embodiments, the plasticizing agent may be presented in an amount that is sufficient to maintain an effective shell plasticity upon migration of a portion of the plasticizing agent from the shell into the fill and/or may be present in a sufficient amount to maintain a desirable dissolution/disintegration profile with respect to the rate and the extent release and/or dispersing of the encapsulated active agent in a specific dissolution medium or upon administration inside the GI tract. The exact amount of plasticizing agent required to compensate for the plasticizing agent anticipated to be lost may depend on a variety of factors, such as the specific fill material and solubility of the plasticizing agent therein. However, those of ordinary skill in the art will be able to readily determine approximate amounts required to maintain effective shell plasticity based on the known characteristics presented by a given dosage form, and will further be able to identify specific amounts through routine experimentation with the dosage form. In one aspect of the invention, such an amount of plasticizing agent may be from about 4% w/w to about 60% w/w of the shell. In another aspect, the amount may be from about 10% w/w to about 35% w/w.

An additional option for maintaining effective shell plasticity and/or a desirable dissolution/disintegration profile of the encapsulated active agent in view of the highly hydrophilic fill material is to include a combination of plasticizing agents in the shell in a total amount sufficient to maintain effective shell plasticity upon migration of a portion of either or both agents into the fill material. In one aspect of the invention, such a combination may include a first plasticizing agent, and a second plasticizing agent having a limited solubility in the fill material as recited above. The total amounts and ratios of each ingredient required to maintain an effective plasticity may be determined by one of ordinary skill in the art in the manners already indicated. While a variety of ratios and amounts are contemplated, in one aspect, the total amount of combined plasticizing agent may be within the ranges already established for plasticizing agents herein.

In addition to the components of a matrix forming material and the at least one plasticizing agent, the shells used in the dosage forms of the present invention may include additional additives as required, in order to achieve a specifically desired formulation or result. Examples of such additives may include without limitation, coloring agents, antioxidants, preservatives, surfactants, and mixtures thereof. Specific amounts of these additives, as well as others not specifically recited will be readily determined by those of ordinary skill in the art, consistent with a working knowledge thereof, and the principles set forth herein.

In certain embodiments, a hydrophobic coating can be used on a surface of the shell. For instance, placing a hydrophobic coating along an inner surface of the shell can prevent or reduce water and plasticizer from migrating into the fill material. Further, placing such a coating on an outer surface of the shell can prevent or reduce the absorption of moisture from the outside environment, and its resultant migration into the fill material. In addition, such coatings can prevent or reduce the migration of plasticizers from the shell and into the fill material.

In the pharmaceutical compositions of the invention, the fill material comprises an enterostatin peptide as described above. In certain embodiments, the fill material can further comprise one or more pharmaceutically acceptable carriers, excipients, or diluents. In certain embodiments, one or more carriers, excipients or diluents can be selected from the non-hygroscopic additives described above.

Exemplary additives include antioxidants, bufferants, antifoaming agents, detackifiers, preservatives, chelating agents, viscomodulators, tonicifiers, flavorants, colorants, odorants, opacifiers, stabilizing agents, solubilizers, binders, fillers, plasticizing agents, lubricants, and mixtures thereof. The specific type and amount of additive may be selected by one of ordinary skill in the art, in order to provide a dosage form with particular characteristics.

One specific lipohilic additive that may be included in the fill material is a triglyceride. In general, these triglycerides are readily available from commercial sources. Examples of suitable triglycerides include vegetable oils, fish oils, animal fats, hydrogenated vegetable oils, partially hydrogenated vegetable oils, medium and long-chain triglycerides, and structured triglycerides. Useful triglycerides include: almond oil; babassu oil; borage oil; blackcurrant seed oil; canola oil; castor oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; partially soy and cottonseed oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; and glyceryl tricaprylate/caprate/stearate. Other useful triglycerides include saturated polyglycolized glycerides (Gelucire 44/14, Gelucire 50/13 and Gelucire 53/10), linoleic glycerides (Maisine 35-I), and caprylic/capric glycerides.

In certain embodiments, the fill material comprises one or more surfactants. Useful surfactants include hydrophilic and lipophilic surfactants. As is well known in the art, the terms “hydrophilic” and “lipophilic” are relative terms. To function as a surfactant, a compound typically includes polar or charged hydrophilic moieties as well as non-polar lipophilic (hydrophobic) moieties. In other words, a surfactant compound must be amphiphilic. An empirical parameter commonly used to characterize the relative hydrophilicity and lipophilicity of non-ionic amphiphilic compounds is the hydrophilic-lipophilic balance (“HLB” value). Surfactants with lower HLB values are more lipophilic, and have greater solubility in oils, while surfactants with higher HLB values are more hydrophilic, and have greater solubility in aqueous solutions.

Using HLB values as a rough guide, hydrophilic surfactants are generally considered to be those compounds having an HLB value of greater than about 10, as well as anionic, cationic, or zwitterionic compounds for which the HLB scale is not generally applicable. Similarly, lipophilic surfactants are compounds having an HLB value of less than about 10.

The hydrophilic surfactant can be any hydrophilic surfactant suitable for use in pharmaceutical compositions. Such surfactants can be anionic, cationic, zwitterionic or non-ionic, although non-ionic hydrophilic surfactants are presently preferred. As discussed above, these non-ionic hydrophilic surfactants will generally have HLB values greater than about 10. Mixtures of hydrophilic surfactants are also within the scope of the invention.

Similarly, the lipophilic surfactant can be any lipophilic surfactant suitable for use in pharmaceutical compositions. In general, suitable lipophilic surfactants will have an HLB value less than about 10. Mixtures of lipophilic surfactants are also within the scope of the invention.

In certain embodiments, the fill material comprises a polyethoxylated fatty acid. Useful hydrophilic surfactants include PEG-8 laurate, PEG-8 oleate, PEG-8 stearate, PEG-9 oleate, PEG-10 laurate, PEG-10 oleate, PEG-12 laurate, PEG-12 oleate, PEG-15 oleate, PEG-20 laurate and PEG-20 oleate. Examples of polyethoxylated fatty acid monoester surfactants commercially available are shown in Table 2.

In certain embodiments, the fill material comprises a PEG fatty acid diesters. Useful hydrophilic surfactants include PEG-20 dilaurate, PEG-20 dioleate, PEG-20 distearate, PEG-32 dilaurate and PEG-32 dioleate.

In general, mixtures of surfactants are also useful in the present invention, including mixtures of two or more commercial surfactant products. Several PEG-fatty acid esters are marketed commercially as mixtures or mono- and diesters. Representative surfactant mixtures include HLB PEG 4-150 mono, dilaurate; PEG 200-6000 mono, dilaurate (Stepan) PEG 4-150 mono, dioleate; PEG 200-6000 mono, dioleate; PEG 4-150 mono, distearate; 200-6000 mono, distearate.

Useful PEG glycerol fatty acid esters include PEG-20 glyceryl laurate, PEG-30 glyceryl laurate, PEG-40 glyceryl laurate, PEG-20 glyceryl oleate, and PEG-30 glyceryl oleate.

A large number of surfactants of different degrees of lipophilicity or hydrophilicity can be prepared by reaction of alcohols or polyalcohols with a variety of natural and/or hydrogenated oils. Most commonly, the oils used are castor oil or hydrogenated castor oil, or an edible vegetable oil such as corn oil, olive oil, peanut oil, palm kernel oil, apricot kernel oil, or almond oil. Preferred alcohols include glycerol, propylene glycol, ethylene glycol, polyethylene glycol, sorbitol, and pentaerythritol. Among these alcohol-oil transesterified surfactants, preferred hydrophilic surfactants are PEG-35 castor oil (Incrocas-35), PEG-40 hydrogenated castor oil (Cremophor RH 40), PEG-25 trioleate (TAGAT® TO), PEG-60 corn glycerides (Crovol M70), PEG-60 almond oil (Crovol A70), PEG-40 palm kernel oil (Crovol PK70), PEG-50 castor oil (Emalex C-50), PEG-50 hydrogenated castor oil (Emalex HC-50), PEG-8 caprylickcapric glycerides (Labrasol), and PEG-6 caprylic/capric glycerides (Softigen 767). Preferred lipophilic surfactants in this class include PEG-5 hydrogenated castor oil, PEG-7 hydrogenated castor oil, PEG-9 hydrogenated castor oil, PEG-6 corn oil (Labrafil® M 2125 CS), PEG-6 almond oil (Labrafil® M 1966 CS), PEG-6 apricot kernel oil (Labrafil® M 1944 CS), PEG-6 olive oil (Labrafil® M 1980 CS), PEG-6 peanut oil (Labrafil® M 1969 CS), PEG-6 hydrogenated palm kernel oil (Labrafil® 2130 BS), PEG-6 palm kernel oil (Labrafil® M 2130 CS), PEG-6 triolein (Labrafil® M 2735 CS), PEG-8 corn oil (Labrafil® WL 2609 BS), PEG-20 corn glycerides (Crovol M40), and PEG-20 almond glycerides (Crovol A40). Also included as oils in this category of surfactants are oil-soluble vitamins, such as vitamins A, D, E, K, etc. Thus, derivatives of these vitamins, such as tocopheryl PEG-1000 succinate (TPGS, available from Eastman), are also suitable surfactants.

Polyglycerol esters of fatty acids are also suitable surfactants for the present invention. Among the polyglyceryl fatty acid esters, preferred lipophilic surfactants include polyglyceryl oleate (Plurol Oleique), polyglyceryl-2 dioleate (Nikkol DGDO), and polyglyceryl-10 trioleate. Preferred hydrophilic surfactants include polyglyceryl-110 laurate (Nikkol Decaglyn 1-L), polyglyceryl-10 oleate (Nikkol Decaglyn 1-O), and polyglyceryl-10 mono, dioleate (Caprol® PEG 860). Polyglyceryl polyricinoleates (Polymuls) are also preferred hydrophilic and lipophilic surfactants. Examples of suitable polyglyceryl esters are shown in Table 7.

Esters of propylene glycol and fatty acids are suitable surfactants for use in the present invention. In this surfactant class, preferred lipophilic surfactants include propylene glycol monolaurate (Lauroglycol FCC), propylene glycol ricinoleate (Propymuls), propylene glycol monooleate (Myverol P-O6), propylene glycol dicaprylate/dicaprate (Captex® 200), and propylene glycol dioctanoate (Captex® 800). Examples of surfactants of this class are given in Table 8.

In general, mixtures of surfactants are also suitable for use in the present invention. In particular, mixtures of propylene glycol fatty acid esters and glycerol fatty acid esters are suitable and are commercially available. One preferred mixture is composed of the oleic acid esters of propylene glycol and glycerol (Arlacel 186). Examples of these surfactants are shown in Table 9.

A particularly useful class of surfactants is the class of mono- and diglycerides. These surfactants are generally lipophilic. Preferred lipophilic surfactants in this class of compounds include glyceryl monooleate, glyceryl ricinoleate, glyceryl laurate, glyceryl dilaurate, glyceryl dioleate, glyceryl mono/dioleate, glyceryl caprylatelcaprate, caprylic acid mono/diglycerides, and mono- and diacetylated monoglycerides.

Sterols and derivatives of sterols are suitable surfactants for use in the present invention. These surfactants can be hydrophilic or lipophilic. Exemplary derivatives include the polyethylene glycol derivatives. An exemplary lipophilic surfactant in this class is cholesterol. An exemplary hydrophilic surfactant in this class is PEG-24 cholesterol ether.

A variety of PEG-sorbitan fatty acid esters are available and are suitable for use as surfactants in the present invention. In general, these surfactants are hydrophilic, although several lipophilic surfactants of this class can be used. Among the PEG-sorbitan fatty acid esters, preferred hydrophilic surfactants include PEG-20 sorbitan monolaurate (Tween-20), PEG-20 sorbitan monopalmitate (Tween-40), PEG-20 sorbitan monostearate (Tween-60), and PEG-20 sorbitan monooleate (Tween-80). Examples of these surfactants are shown in Table 12.

Ethers of polyethylene glycol and alkyl alcohols are suitable surfactants for use in the present invention. Useful lipophilic ethers include PEG-3 oleyl ether (Volpo 3) and PEG-4 lauryl ether (Brij 30).

Esters of sugars are suitable surfactants for use in the present invention. Useful hydrophilic surfactants in this class include sucrose monopalmitate and sucrose monolaurate.

Polyoxyethylene-polyoxypropylene block copolymers are also suitable for use in the present invention. These surfactants are available under various trade names, including Synperonic PE series (ICI); Pluronic® series (BASF), Emkalyx, Lutrol (BASF), Supronic, Monolan, Pluracare, and Plurodac. A generic term for these polymers is “ploxamer” (CAS 9003-11-6). Useful surfactants of this class include Poloxamers 108, 188, 217, 238, 288, 338, and 407. Preferred lipophilic surfactants in this class include Poloxamers 124, 182, 183, 212, 331, and 335.

Sorbitan esters of fatty acids are suitable surfactants for use in the present invention. Among these esters, preferred lipophilic surfactants include sorbitan monolaurate (Arlacel 20), sorbitan monopalmitate (Span-40), sorbitan monooleate (Span-80), sorbitan monostearate, and sorbitan tristearate.

Esters of lower alcohols (C₂ to C₄) and fatty acids (C₈ to C₁₈) are suitable surfactants for use in the present invention. Among these esters, preferred lipophilic surfactants include ethyl oleate (Crodamol EO), isopropyl myristate (Crodamol IPM), and isopropyl palmitate (Crodamol IPP).

Ionic surfactants, including cationic, anionic and zwitterionic surfactants, are suitable hydrophilic surfactants for use in the present invention. Preferred anionic surfactants include fatty acid salts and bile salts. Specifically, preferred ionic surfactants include sodium oleate, sodium lauryl sulfate, sodium lauryl sarcosinate, sodium dioctyl sulfosuccinate, sodium cholate, and sodium taurocholate.

A hydrophilic surfactant can also be, or include as a component, an ionic surfactant. Preferred ionic surfactants include alkyl ammonium salts; bile acids and salts, analogues, and derivatives thereof; fusidic acid and derivatives thereof; fatty acid derivatives of amino acids, oligopeptides, and polypeptides; glyceride derivatives of amino acids oligopeptides, and polypeptides; acyl lactylates; mono-diacetylated tartaric acid esters of mono-diglycerides; succinylated monoglycerides; citric acid esters of mono-diglycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate; carnitines; and mixtures thereof. More preferable ionic surfactants include bile acids and salts, analogues, and derivatives thereof; lecithins, lysolecithin, phospholipids, lysophospholipids and derivatives thereof; salts of alkylsulfates; salts of fatty acids; sodium docusate; acyl lactylates; mono-diacetylated tartaril acid esters of mono-diglycerides; succinylated monoglycerides; citric acid esters of mono-diglycerides; camitines; and mixtures thereof.

More specifically, useful ionic surfactants include lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholate, taurocholate, glycocholate, deoxycholate, taurodeoxycholate, chenodeoxycholate, glycodeoxycholate, glycochenodeoxycholate, taurochenodeoxycholate, ursodeoxycholate, tauroursodeoxycholate, glycoursodeoxycholate, cholylsarcosine, N-methyl taurocholate, caproate, caprylate, caprate, laurate, myristate, palmitate, oleate, ricinoleate, linoleate, linolenate, stearate, lauryl sulfate, teracecyl sulfate, docusate, lauroyl camitines, palmitoyl camitines, myristoyl carnitines, and salts and mixtures thereof.

The carrier of the present compositions may include a combination of at least two surfactants, at least one of which is hydrophilic. In one embodiment, the present invention includes at two surfactants that are hydrophilic, and useful hydrophilic surfactants are listed above. In certain embodiments, the carrier includes at least one hydrophilic surfactant and at least one lipophilic surfactant.

Useful lipophilic surfactants include alcohols; polyoxyethylene alkylethers; fatty acids; glycerol fatty acid esters; acetylated glycerol fatty acid esters; lower alcohol fatty acids esters; polyethylene glycol fatty acids esters; polyethylene glycol glycerol fatty acid esters; polypropylene glycol fatty acid esters; polyoxyethylene glycerides; lactic acid derivatives of mono/diglycerides; propylene glycol diglycerides; sorbitan fatty acid esters; polyoxyethylene sorbitan fatty acid esters; polyoxyethylene-polyoxypropylene block copolymers; transesterified vegetable oils; sterols; sterol derivatives; sugar esters; sugar ethers; sucroglycerides; polyoxyethylene vegetable oils; and polyoxyethylene hydrogenated vegetable oils.

As with the hydrophilic surfactants, lipophilic surfactants can be reaction mixtures of polyols and fatty acids, glycerides, vegetable oils, hydrogenated vegetable oils, and sterols.

Specifically useful lipophilic surfactants include myristic acid; oleic acid; lauric acid; stearic acid; palmitic acid; PEG 1-4 stearate; PEG 2-4 oleate; PEG-4 dilaurate; PEG-4 dioleate; PEG-4 distearate; PEG-6 dioleate; PEG-6 distearate; PEG-8 dioleate; PEG 3-16 castor oil; PEG 5-10 hydrogenated castor oil; PEG 6-20 corn oil; PEG 6-20 almond oil; PEG-6 olive oil; PEG-6 peanut oil; PEG-6 palm kernel oil; PEG-6 hydrogenated palm kernel oil; PEG-4 capric/caprylic triglyceride, mono, di, tri, tetra esters of vegetable oil and sorbitol; pentaerythrityl di, tetra stearate, isostearate, oleate, caprylate, or caprate, polyglyceryl 2-4 oleate, stearate, or isostearate; polyglyceryl 4-10 pentaoleate; polyglyceryl-3 dioleate; polyglyceryl-6 dioleate; polyglyceryl-10 trioleate; polyglyceryl-3 distearate; propylene glycol mono- or diesters of a C₆ to C₂₀ fatty acid; monoglycerides of C₆ to C₂₀ fatty acids; acetylated monoglycerides of C₆ to C₂₀ fatty acids; diglycerides of C₆ to C₂₀ fatty acids; lactic acid derivatives of monoglycerides; lactic acid derivatives of diglycerides; cholesterol; phytosterol; PEG 5-20 soya sterol; PEG-6 sorbitan tetra, hexastearate; PEG-6 sorbitan tetraoleate; sorbitan monolaurate; sorbitan monopalmitate; sorbitan mono, trioleate; sorbitan mono, tristearate; sorbitan monoisostearate; sorbitan sesquioleate; sorbitan sesquistearate; PEG 2-5 oleyl ether; POE 2-4 lauryl ether; PEG-2 cetyl ether; PEG-2 stearyl ether; sucrose distearate; sucrose dipalmitate; ethyl oleate; isopropyl myristate; isopropyl palmitate; ethyl linoleate; isopropyl linoleate; and poloxamers.

If desired, the pharmaceutical compositions of the present invention can optionally include additional compounds to enhance the solubility of the therapeutic agent or the triglyceride in the composition. Examples of such compounds, referred to as “solubilizers,” include: alcohols and polyols, such as ethanol, isopropanol, butanol, benzyl alcohol, ethylene, glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene zlycols having an average molecular weight of about 200 to about 6000, such as tetrahydrofurfuryl alcohol PEG ether (glycofurol, available commercially from BASF under the trade name Tetraglycol) or methoxy PEG (Union Carbide); amides, such as 2-pyrrolidone, 2-piperidone, 6-caprolactam, N-alkylpyrrolidone, N-hydroxyalkylpyrrolidone, N-alkylpiperidone, N-alkylcaprolactam, dimethylacetamide, and polyvinylpyrrolidone; esters, such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, phi-caprolactone and isomers thereof, delta-valerolactone and isomers thereof, .beta.-butyrolactone and isomers thereof; and other solubilizers known in the art, such as dimethyl acetamide, dimethyl isosorbide (Arlasolve DMI (ICI)), N-methylpyrrolidones (Pharmasolve (ISP)), monooctanoin, diethylene glycol monoethyl ether (available from Gattefosse under the trade name Transcutol), and water.

The formulations of the present invention optionally include one or more stabilizing agents to increase the stability and/or compatibility of the composition when formulated into a dosage form. Suitable stabilizing agents include suspending agents, flocculating agents, thickening agents, gelling agents, buffering agents, antioxidants, preservatives, antimicrobial agents, and mixtures thereof.

A useful stabilizing agent in most cases is a suspending agent that imparts increased viscosity and retards sedimentation, to prevent caking. A wide variety of pharmaceutically acceptable excipient with such attributes, of the many well known in the art, can be used as such a suspending agent. Suitable suspending agents include cellulose derivatives, clays, natural gums, synthetic gums, or other agents known in the art. Specific suspending agents, by way of example, include without limitation, microcrystalline cellulose, sodium carboxymethylcellulose, powdered cellulose, ethymethylcellulose, hydroyxypropyl methylcellulose, methylcellulose, ethylcellulose, ethylhydroxy ethylcellulose, hydroxypropyl cellulose, attapulgite, bentonite, hectorite, montmorillonite, silica gel, fumed silicon dioxide, colloidal silicon dioxide, acacia, agar, carrageenan, guar gum, locust bean gum, pectin, sodium alginate, propylene glycol alginate, tamarind gum, xanthan gum, carbomer, povidone, sodium starch glycolate, starches, tragacanth, magnesium aluminum silicate, aluminum silicate, magnesium silicate, gelatin, and glycyrrhizin. These suspending agents can further impart different flow properties to the suspension. The flow properties of the suspension can be Newtonian, plastic, pseudoplastic, thixotropic or combinations thereof. Mixtures of suspending agents may also be used to optimize flow properties and viscosity.

The stabilizing agent may also be a flocculating agent that enables particles to associate in loose aggregates or “flocs.” Although these flocs may settle rapidly, they are easily redispersed. Many flocculating agents known in the art can be utilized, including surfactants, hydrophilic polymers, clays, and electrolytes. Any other pharmaceutically acceptable exicipient with such attributes can also be utilized as a flocclulating agent. In some cases, the flocculating agent may serve a dual purpose, serving not only as a stabilizing agent but also, for example, as a component of the solid particles or as a suspending agent. Suitable flocculating agents include, but are not limited to, sodium lauryl sulfate, sodium docusate, benzalkonium chloride, polysorbate 80, sorbitan monolaurate, sodium carboxymethylcellulose, xanthan gum, tragacanth, methylcellulose, magnesium aluminum silicate, attapulgite, bentonite, potassium dihydrogen phosphate, aluminum chloride, and sodium chloride. The formulation may include both a flocculating agent and a suspending agent, so that the sendimentation of flocs can be retarded.

The stabilizing agent may also be a thickening agent, selected to increase the viscosity of the suspension to a degree sufficient to reduce and retard sedimentation of suspended active agent particles. Any pharmaceutically acceptable excipient with such attributes can be used in the present invention. Typically, compounds that soften slightly above ambient temperature are desirable for this purpose. Preferred thickening agents have a melting point greater than about 25.degree. C., and can be reversibly liquified and solidified. With an appropriate amount of such a thickening agent, the formulation as a whole can acquire this thermosoftening property.

Other additives conventionally used in pharmaceutical compositions can be included, and these additives are well known in the art. Such additives include detackifiers, anti-foaming agents, buffering agents, antioxidants, preservatives, chelating agents, viscomodulators, tonicifiers, flavorants, colorants odorants, opacifiers, binders, fillers, plasticizers, lubricants, and mixtures thereof. The amounts of such additives can be readily determined by one skilled in the art, according to the particular properties desired.

The pharmaceutical composition of the invention can be in any dosage form known to those of skill in the art for an encapsulated composition. Useful dosage forms include the basic elements as recited herein such as a fill material and a shell encapsulating the fill material. One general category of such dosage form specifically contemplated to be within the scope of the present invention is capsules.

A wide variety of capsules, including methods and materials for the making thereof, are known to those of ordinary skill in the art, such as hard and soft capsules that are either single piece or two piece capsules. Many typical capsules of this nature provide an instant release of the active agent and thus release substantially all of the active agent in a relatively short time period. However, additional steps may be taken to prolong or extend release of the active agent, for example, by adding a coating to the capsule to provide a sustained release formulation. A variety of such coatings are known to those of ordinary skill in the art, such as enteric and osmotic coatings, as well as a number of other mechanisms for prolonging or otherwise altering release of the active agent from the capsule in a desired manner.

Additionally, when two piece capsules are used, a number of techniques are known for banding or sealing the pieces of the capsule together to prevent leakage of the encapsulated fill material. Such processes and techniques may be used in connection with the dosage forms of the present invention, when such dosage forms involve a two piece capsule.

Accordingly, in one aspect, the dosage form of the present invention may be a capsule. In another aspect, the capsule may be a gelatin capsule. In yet another aspect, the gelatin capsule may be a soft gelatin capsule. In a further aspect, the capsule may be a single piece capsule. In an additional aspect, the capsule may be a two piece capsule which is banded or sealed in order to prevent leakage of the encapsulated fill material. In another aspect, the capsule may be an instant release formulation. In a further aspect, the capsule may include one or more mechanisms for varying or sustaining the release of the active agent.

4.6 Solid Dispersions

In further embodiments, the present invention provides a non-hygroscopic pharmaceutical composition comprising a non-hygroscopic solid dispersion of an enterostatin peptide. Suitable solid dispersions include those that comprise a matrix forming agent, one or more optional fillers and the enterostatin peptide.

In certain embodiments, the non-hygroscopic solid dispersion is sufficient to yield a non-hygroscopic pharmaceutical composition of the invention that absorbs less than 10%, 9%, 8%, 7%, 6%, 5%, 4%, 3%, 2% or 1% water by weight in an atmosphere of normal humidity. In certain embodiments, the non-hygroscopic solid dispersion is sufficient to yield a non-hygroscopic pharmaceutical composition of the invention that will remain solid for at least 1, 2, 3, 4, 5, 10, 15 or 20 days at least 25%, 50% or 75% humidity. In preferred embodiments, the non-hygroscopic solid dispersion is sufficient to yield a non-hygroscopic pharmaceutical composition of the invention that will remain solid for at least 4 or 10 days at at least 58% humidity. In certain embodiments, the non-hygroscopic solid dispersion is sufficient to yield a non-hygroscopic pharmaceutical composition of the invention that will gain less than 35%, 30%, 25% or 20% water, by weight, when moved from 5% to 95% relative humidity under techniques known to those of skill in the art. In certain embodiments, the non-hygroscopic solid dispersion is sufficient to yield a non-hygroscopic pharmaceutical composition of the invention that will lose less than 35%, 30%, 25% or 20% water, by weight, when moved from 95% to 5% relative humidity under techniques known to those of skill in the art. In certain embodiments, the non-hygroscopic solid dispersion is sufficient to yield a non-hygroscopic pharmaceutical composition of the invention that will gain less than 35%, 30%, 25% or 20% water, by weight, when moved from 5% to 95% relative humidity, and that will lose less than 35%, 30%, 25% or 20% water, by weight, when moved from 95% to 5% relative humidity.

The term “matrix forming agent” herein refers to a polymer that itself or in combination with a filler and/or any other excipient or excipients, is able to create a matrix wherein the enterostatin peptide can be dispersed or dissolved. The matrix forming agent can be any matrix forming agent capable of forming a solid dispersion known to those of skill in the art. In certain embodiments, the matrix forming agent can be selected from the group consisting of hydroxyethylcellulose, HPC, HPMC, HPMC phthalate, PVP, PEG, polyglycolized glycerides, cyclodextrins and carbomers. The composition can further comprise one or more pharmaceutically acceptable carriers, diluents or excipients known to those of skill in the art.

The ratio of enterostatin to matrix forming agent can be any ratio that yields a non-hygroscopic composition. In certain embodiments, the ratio is within the range of 10:1 to 1:10, 5:1 to 1:5, 4:1 to 1:4, or 2:1 to 1:1 matrix forming agent to enterostatin, or the range is about 1:1. In particular embodiments the ratio is about 10:1, 5:1, 4:1, 3:1, 2.5:1, 2:1, 1:1, 1:2.5, 1:3, 1:4, 1:5, or 1:10 matrix forming agent to enterostatin.

The matrix forming agent can be present in any amount in the composition that is sufficient to yield a non-hygroscopic composition. In certain embodiments, the matrix forming agent is present in an amount of from about 1% to about 90% of the weight of the final composition, of from about 10% to about 90% of the weight of the final composition, of from about 20% to about 90% of the weight of the final composition, of from about 25% to about 90% of the weight of the final composition or of from about 50% to about 90% of the weight of the final composition. In particular embodiments, the matrix forming agent is present in an amount of about 10%, 15%, 20%, 25%, 30%, 33%, 40%, 50%, 60%, 67%, 70%, 75%, 80%, 85% or 90% of the final composition. In preferred embodiments, the matrix forming agent or agents are present in an amount sufficient to form a solid dispersion under conditions apparent to those of skill in the art.

Compositions of the present invention may also optionally include other therapeutic ingredients, anti-caking agents, preservatives, sweetening agents, colorants, flavors, desiccants, plasticizers, dyes, and the like. Such optional ingredients are described in the sections below.

In one embodiment, the matrix forming agent is a hydroxypropylcellulose. Exemplary hydroxypropylcelluloses useful in the present invention include those having low dynamic viscosity in aqueous media, preferably below about 400 cps, e.g., below about 150 cps as measured in a 2% aqueous solution at 25° C. Preferred hydroxypropylcelluloses have a low degree of substitution, and an average molecular weight below about 200,000 daltons, e.g., from about 50,000 to about 150,000 daltons. HPC is commercially available, for example, under the trade names Klucel™ LF, Klucel™ EF and Klucel™ JF (Aqualon), and NiSSO™ HPC-L (Nippon Soda).

In another embodiment, the matrix forming agent is a cyclodextrin, for example a β-cyclodextrin or an α-cyclodextrin. Examples of suitable β-cyclodextrins include methyl-β-cyclodextrin, dimethyl-β-cyclodextrin, hydroxypropyl-β-cyclodextrin (HPBCD), glycosyl-β-cyclodextrin, maltosyl-β-cyclodextrin, sulfo-β-cyclodextrin and sulfo-alkylethers, e.g., sulfo-C₁₋₄-alkylethers, of β-cyclodextrin. Examples of α-cyclodextrins include glucosyl-α-cyclodextrin and maltosyl-α-cyclodextrin.

In another embodiment, the matrix forming agent is a polyglycolized glyceride. Polyglycolized glycerides are generally mixtures of monoesters, diesters and triesters of glycerol with monoesters and diesters of polyethylene glycols having a average molecular weight of about 200 and 6000. They can be obtained by partial transesterification of triglycerides with polyethylene glycol or by esterification of glycerol and polyethylene glycol with fatty acids using known reactions. Preferably, such fatty acids have 8-22, more preferably 8-18, carbon atoms. Examples of natural vegetable oils, which may be used as a source of such fatty acids, include palm kernel oil and palm oil. The polyethylene glycol can optionally be replaced with another polyol, for example a polyglycerol or sorbitol. Polyglycolized glycerides are available for example under the trade name Gelucire® (Gattefosse).

In another embodiment, the matrix forming agent is hydroxyethylcellulose. Exemplary hydroxyethylcelluloses useful in the invention include those having low dynamic viscosity in aqueous media, preferably below about 400 cps, e.g., below about 150 cps as measured in a 2% aqueous solution at 25° C. Hydroxyethylcellulose is available for example under the trade names Cellosize™. (Amerchol) and Natrusol™ (Aqualon).

In another embodiment, the matrix forming agent is a carbomer. Carbomers are high molecular weight polymers of acrylic acid that are cross-linked with either allylsucrose or allyl esters of pentaerythritol. Carbomers are available, for example, under the trade name Carbol™ (Noveon Pharmaceuticals).

In another preferred embodiment, the matrix forming agent is hydroxypropylmethylcellulose. In certain embodiments, the hydroxypropylmethylcellulose has a low apparent dynamic viscosity, preferably below about 100 cps as measured at 20° C. for a 2% by weight aqueous solution, more preferably below about 50 cps, most preferably below about 20 cps, for example 3 cps. Hydroxypropylmethylcellulose, including a grade having apparent dynamic viscosity of 3 cps, is available for example under the trade name Pharmacoat™ 603 (Shin-Etsu). In another embodiment, the matrix forming agent is hydroxypropylmethylcellulose phthalate, which is available for example from Shin-Etsu.

In yet another embodiment, the matrix forming agent is povidone. Povidone is available for example under the trade names Plasdone™ (ISP) and Kollidon™ (BASF). Povidone having an average molecular weight of about 8,000 to about 50,000 daltons is useful.

In another embodiment, the matrix forming agent is a PEG that is solid at ambient temperatures. Such PEGs include those that have an average molecular weight of about 1,000 daltons to about 35,000 daltons, for example about 8,000 daltons. PEG is available for example under the trade name Carbowax™ (Dow).

The term “filler” or “fill material” herein refers to inert materials that serve to increase the mass and/or bulk density of the solid dispersion, so that, for example, the solid dispersion can be relatively easily incorporated into a conventional dosage form, e.g., a tablet or capsule. Fillers contemplated for use in the present invention include for example microcrystalline cellulose, lactose, calcium carbonate, carboxymethylcellulose calcium, carboxymethylcellulose sodium, dibasic calcium phosphate dihydrate, tribasic calcium phosphate, calcium sulfate, dextrose, ethyl cellulose, fructose, kaolin, magnesium carbonate, magnesium stearate, magnesium trisilicate, maltol, maltodextrin, mannitol, methyl cellulose, powdered cellulose, pregelatinized starch, starch, sterilizable maize starch, compressible sugar, confectioner's sugar and the like. Preferably the filler used does not adversely affect the stability and/or dissolution performance of the dispersion.

In certain embodiments, a composition of the present invention can comprise a hygroscopic or deliquescent filler. Preferably, the hygroscopic or deliquescent filler is present in an amount that does not increase the hygroscopicity of the overall composition beyond the limits desired by the practitioner in the art. Suitable hygroscopic and/or deliquescent fillers include for example microcrystalline cellulose, tribasic calcium phosphate, anhydrous calcium sulfate, carboxymethylcellulose calcium, carboxymethylcellulose sodium, anhydrous dextrose, fructose, anhydrous lactose, anhydrous magnesium stearate, magnesium trisilicate, maltodextrin, methylcellulose, powdered cellulose, pregelatinized starch, starch, sterilizable maize starch, compressible sugar, confectioner's sugar and the like.

Preferably the filler is present in an amount sufficient to enable the solid dispersion, once formed, to be in a flowable state, such as a powder, that can be easily incorporated into conventional dosage forms, such as tablets and capsules. Accordingly, the filler is generally present in an amount of about 1% to about 95%, preferably about 5% to about 30% by weight of the composition.

If desired, the carrier medium can further comprise other pharmaceutically acceptable excipients selected, for example, from antioxidants such as α-tocopherol, ascorbic acid, ascorbyl palmitate, butylated hydroxyanisole and butylated hydroxytoluene; disintegrants such as sodium starch glycolate and sodium starch fumarate; flavoring agents such as aspartame, saccharin and saccharin sodium; glidants such as magnesium aluminum silicate, talc and titanium dioxide; lubricants such as stearic acid; neutralizing agents such as dibasic sodium phosphate and monobasic sodium phosphate; preservatives; stabilizers; surfactants such as docusate sodium and sorbitan esters; wetting agents such as poloxamers and sodium lauryl sulfate; and thickeners and coatings such as gelatin and polymethacrylates. Such excipients can alternatively or additionally be later blended with the solid dispersion, once it has formed, prior or subsequent to incorporation into a pharmaceutical dosage form.

4.7 Preparation of Compounds and Compositions of the Invention

The compositions can be prepared according to any method known to those of skill in the art, including those illustrated in the examples below.

Enterostatin can be prepared according to any technique apparent to those of skill. Exemplary techniques for the preparation of enterostatin are described in U.S. Pat. No. 5,494,894, the contents of which are hereby incorporated by reference in their entirety. In certain embodiments, enterostatin can be prepared synthetically, for example by solution phase or solid phase peptide synthesis. See Merrifield, 1963, J. Am. Chem. Soc. 85:2149; Fields et al., 1990, Int J Pept Protein Res. 35:161-214; Fields et al., 1991, Pept Res. 4:95-101; the contents of which are hereby incorporated by reference in their entireties. In further embodiments, enterostatin can be obtained from natural sources, recombinant sources or commercial sources.

Although the final compositions of the invention can have reduced hygroscopicity, preparation of the compositions themselves can be advantageous to reduce the amount of water in the final form. Accordingly, in some embodiments, the compositions is prepared under anhydrous conditions. However, the present invention is in no way limited by the method of preparation of the compositions. Accordingly, the present invention also provides methods of preparing the compositions without regard to hydrous or anhydrous conditions.

Once prepared, the enterostatin compositions can be stored under any conditions for the storage of a peptide complex known to those of skill in the art. Although the compositions can display advantageous hygroscopicity, in preferred embodiments the compositions are stored at low humidity conditions to maximize the stability of the compositions.

Solid dispersions of the invention can be prepared by any suitable process. Known methods of preparing solid dispersions include solvent, fusion, or fusion-solvent methods as described in standard reference texts, such as Habib (2001), Pharmaceutical Solid Dispersions, Technomic Publishing Co., Lancaster, Pa., the content of which is incorporated by reference in their entirety. The processes described below are presented for illustrative purposes, and are not intended to limit the scope of the invention.

In one embodiment, a solid dispersion is prepared according to the solvent method, by dissolving a matrix forming agent, a filler and a hygroscopic and/or deliquescent drug in a solvent. Solvents contemplated for use in this process include water; alcohols such as methanol, ethanol and isopropanol; esters such as ethyl acetate; ethers such as diethyl ether; ketones such as acetone; halogenated hydrocarbons such as dichloroethane; and combinations thereof such as a mixture of ethanol and acetone. The solvent is then evaporated, for example using elevated temperature and/or a vacuum, or by freeze drying or spray drying. As the solvent evaporates, supersaturation occurs, followed by simultaneous precipitation of both the matrix forming agent and the drug in solid form. The resulting precipitate, which has the drug dissolved or suspended in a carrier medium formed from the matrix forming agent and the filler, is then dried to produce a solid dispersion of the invention. This process is especially useful for drugs that are soluble in the carrier medium selected and for drugs that are thermolabile.

In another embodiment, a solid dispersion is prepared according to the fusion method, wherein a matrix forming agent is heated to a temperature above its melting point and a hygroscopic and/or deliquescent drug is added with mixing to the melted agent. A filler is either heated along with the matrix forming agent or incorporated along with the drug by mixing after the melting of the matrix forming agent. The resulting composition is then cooled, for example allowed to cool naturally, with constant mixing, e.g., by stirring, to produce a formulation that is a solid dispersion having the drug evenly dispersed therein. If the drug is soluble in the matrix forming agent, it remains dissolved in the formulation, which is therefore a solid solution or molecular dispersion. If the drug is not soluble in the matrix forming agent, it is dispersed in crystalline or amorphous particulate form in the solid dispersion.

In yet another embodiment, a solid dispersion is prepared according to the fusion-solvent method, wherein a matrix forming agent is heated until melted and a solution of a hygroscopic and/or deliquescent drug in a suitable solvent is added with mixing thereto. Again, a filler is either heated along with the matrix forming agent or is incorporated along with the drug by mixing after the melting of the matrix forming agent. If, upon cooling, the resulting composition is capable of holding a certain proportion of solvent while maintaining its solid properties, and if the solvent is innocuous, the need for solvent removal is eliminated; otherwise, the solvent is removed, for example using elevated temperature and/or a vacuum, or by freeze drying or spray drying.

4.8 Filler Materials, Excipients, Diluents, Carriers

The above compositions of the present invention may further include any conventional pharmaceutically acceptable filler, excipient, diluent or carrier known to those of skill in the art. Preferably, the additional material should not increase the hygroscopicity of the composition beyond the limits desired by the practitioner of skill in the art.

Examples of excipients for use as the pharmaceutically acceptable carriers and the pharmaceutically acceptable inert carriers and the aforementioned additional ingredients include, but are not limited to those that follow.

Binders are agents used to impart cohesive qualities to the powdered material. Binders impart a cohesiveness to the tablet formulation which insures the tablet remains intact after compression, and improves the free-flowing qualities by the formulation of granules of desired hardness and size. Suitable binder materials include, but are not limited to, starch (including corn starch and pregelatinzed starch), gelatin, sugars (including sucrose, glucose, dextrose, lactose and sorbitol), polyethylene glycol, waxes, natural and synthetic gums, e.g., acacia, tragacanth, sodium alginate, celluloses, and Veegum, and synthetic polymers such as polymethacrylates and polyvinylpyrrolidone.

Lubricants have a number of function in tablet manufacture. They prevent adhesion of the tablet material to the surface of the dies and punches, reduce interparticle friction, facilitate the ejection of the tablets from the die cavity and may improve the rate of flow of the tablet granulation. Examples of suitable lubricants include, but are not limited to, magnesium stearate, calcium stearate, stearic acid, glyceryl behenate, talc, sodium lauryl sulfate, sodium stearyl fumarate, polyethylene glycol or mixtures thereof. A preferred lubricant herein is magnesium stearate.

Preferably, the lubricant is present in an amount from about 0.25% to about 5% of the weight of the final composition and more preferably from about 0.5 to about 1.5% of the weight of the final composition.

A disintegrant is a substance, or a mixture of substances, added to a tablet to facilitate its breakup or disintegration after administration. Materials serving as disintegrants have been classified chemically as starches, clay, celluloses, aligns, gums and cross-linked polymers. Examples of suitable disintegrants include, but are not limited to, crosscarmelose sodium, sodium starch glycolate, starch, magnesium aluminum silicate, colloidal silicon dioxide, methylcellulose, agar, bentonite, alginic acid, guar gum, citrus pulp, carboxymethyl cellulose, microcrystalline cellulose, or mixtures thereof. A preferred disintegrant is sodium starch glycolate.

Preferably, the disintegrant is present in an amount from about 0.5% to about 25% of the weight of the final composition and more preferably from about 1% to about 15% of the weight of the final composition.

Glidants are substances which improve the flow characteristics of a powder mixture. Examples of glidants include, but are not limited to colloidal silicon dioxide, talc or mixtures thereof.

Preferably, the glidant is present in an amount of from about 0.1% to about 10% of the weight of the final composition and more preferably from 5 about 0.1% to about 5% of the weight of the final composition.

The adsorbent may be, for example colloidal silicon dioxide, microcrystalline cellulose, calcium silicate or mixtures thereof.

Preferably, the adsorbent is present in an amount from about 0.05% to about 42% of the weight of the final composition and more preferably from about 0.05% to about 37% of the weight of the final composition.

If desired, other ingredients, such as diluents, stabilizers and antiadherants, which are conventionally used for pharmaceutical formulations, may be included in the present formulations.

Optional ingredients include coloring and flavoring agents which are well known in the art.

Useful fillers include talc, calcium carbonate (e.g., granules or powder), dibasic calcium phosphate, tribasic calcium phosphate, calcium sulfate (e.g., granules or powder), microcrystalline cellulose, powdered cellulose, dextrates, kaolin, mannitol, silicic acid, sorbitol, starch, pre-gelatinized starch, or mixtures thereof.

Useful anticaking agents include calcium silicate, magnesium silicate, silicon dioxide, colloidal silicon dioxide, talc, or mixtures thereof.

Useful antimicrobial agents include benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenylmercuric acetate, phenylmercuric nitrate, potassium sorbate, propylparaben, sodium benzoate, sodium dehydroacetate, sodium propionate, sorbic acid, thimersol, thymo, or mixtures thereof.

Useful coating agents include sodium carboxymethyl cellulose, cellulose acetate phthalate, ethylcellulose, gelatin, pharmaceutical glaze, hydroxypropyl cellulose, hydroxypropyl methylcellulose, hydroxypropyl methyl cellulose phthalate, methylcellulose, polyethylene glycol, polyvinyl acetate phthalate, shellac, sucrose, titanium dioxide, carnuba wax, microcrystalline wax, or mixtures thereof.

In a preferred embodiment, a composition of the invention is a pharmaceutical composition or a single unit dosage form. Pharmaceutical compositions and single unit dosage forms of the invention comprise a prophylactically or therapeutically effective amount of one or more prophylactic or therapeutic agents (e.g., a composition of the invention, or other prophylactic or therapeutic agent), and a typically one or more pharmaceutically acceptable carriers or excipients. In a specific embodiment and in this context, the term “pharmaceutically acceptable” means approved by a regulatory agency of the Federal or a state goverment or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typical pharmaceutical compositions and dosage forms comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a patient and the specific active ingredients in the dosage form. The composition or single unit dosage form, if desired, can also contain minor amounts of wetting or emulsifying agents, or pH buffering agents.

Lactose-free compositions of the invention can comprise excipients that are well known in the art and are listed, for example, in the U.S. Pharmocopia (USP) SP (XXI)/NF (XVI). In general, lactose-free compositions comprise an active ingredient, a binder/filler, and a lubricant in pharmaceutically compatible and pharmaceutically acceptable amounts. Preferred lactose-free dosage forms comprise an active ingredient, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

The pharmaceutical compositions and single unit dosage forms can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a prophylactic or therapeutic agent preferably in purified form, together with a suitable amount of carrier so as to provide the form for proper administration to the patient. The formulation should suit the mode of administration. In a preferred embodiment, the pharmaceutical compositions or single unit dosage forms are sterile and in suitable form for administration to a subject, preferably an animal subject, more preferably a mammalian subject, and most preferably a human subject.

A pharmaceutical composition of the invention is formulated to be compatible with its intended route of administration. Examples of routes of administration include, but are not limited to, parenteral, e.g., intravenous, intradermal, subcutaneous, intramuscular, subcutaneous, oral, buccal, sublingual, inhalation, intranasal, transdermal, topical, transmucosal, intra-tumoral, intra-synovial and rectal administration. In a specific embodiment, the composition is formulated in accordance with routine procedures as a pharmaceutical composition adapted for intravenous, subcutaneous, intramuscular, oral, intranasal or topical administration to human beings. In an embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of inflammation or a related disorder may contain larger amounts of one or more of the active ingredients it comprises than a dosage form used in the chronic treatment of the same disease. Also, the therapeutically effective dosage form may vary among different types of cancer. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Typical dosage forms of the invention comprise a composition of the invention, or a pharmaceutically acceptable salt, solvate or hydrate thereof lie within the range of from about 0.1 mg to about 1000 mg per day, given as a single once-a-day dose in the morning but preferably as divided doses throughout the day taken with food. Particular dosage forms of the invention have about 0.1, 0.2, 0.3, 0.4, 0.5, 1.0, 2.0, 2.5, 5.0, 10.0, 15.0, 20.0, 25.0, 50.0, 100, 200, 250, 500 or 1000 mg of the active enterostatin.

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

In preferred embodiments, the oral dosage forms are solid and prepared under anhydrous conditions with anhydrous ingredients, as described in detail in the sections above. However, the scope of the invention extends beyond anhydrous, solid oral dosage forms. As such, further forms are described herein.

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

4.9 Methods of Treatment or Prevention

The enterostatin compositions of the invention can be used for the treatment or prevention of any disorder or condition amenable to treatment with enterostatin according to the judgment of those of skill in the art. The condition can be associated with normal or abnormal enterostatin function. For instance, in certain embodiments, an enterostatin composition of the invention can be administered to a subject that expresses or secretes a low amount of enterostatin to reduce or ameliorate any symptom of the low amount of enterostatin. Such methods of treatment are described in U.S. provisional application No. 60/750,206, filed Dec. 13, 2005, the contents of which are hereby incorporated by reference in their entirety.

In certain embodiments, the compositions of the invention can be used for the treatment or prevention of overweight, obesity, metabolic disorders, hypertension, lipid related disorders, and type II diabetes.

4.10 Dosage & Frequency of Administration

The amount of the composition of the invention which will be effective in the prevention, treatment, management, or amelioration of a disorder or one or more symptoms thereof will vary with the nature and severity of the disease or condition, and the route by which the active ingredient is administered. The frequency and dosage will also vary according to factors specific for each patient depending on the specific therapy (e.g., therapeutic or prophylactic agents) administered, the severity of the disorder, disease, or condition, the route of administration, as well as age, body, weight, response, and the past medical history of the patient. Effective doses may be extrapolated from dose-response curves derived from in vitro or animal model test systems. Suitable regiments can be selected by one skilled in the art by considering such factors and by following, for example, dosages reported in the literature and recommended in the Physician's Desk Reference (59 h ed., 2005)

Exemplary doses of a composition include milligram or microgram amounts of the active peptide per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). For composition of the invention, the dosage administered to a patient is typically 0.01 mg/kg to 15 mg/kg of the patient's body weight, based on weight of the active peptide. Preferably, the dosage administered to a patient is between 0.01 mg/kg and 15 mg/kg, 0.01 mg/kg and 10 mg/kg, 0.01 mg/kg and 5 mg/kg, 0.01 and 4 mg/kg, 0.01 and 3 mg/kg, 0.01 mg/kg and 2 mg/kg, 0.01 mg/kg and 1 mg/kg, 0.02 mg/kg and 1 mg/kg, 0.10 mg/kg and 2.5 mg/kg, of the patient's body weight.

In general, the recommended daily dose range of a composition of the invention for the conditions described herein lie within the range of from about 0.01 mg to about 1000 mg of the active peptide per day, as a single dose or multiple doses per day. Specifically, a total daily dose range should be from about 1 mg to about 500 mg per day, more specifically, between about 10 mg and about 200 mg per day. In managing the patient, the therapy can be initiated at a lower dose, perhaps about 1 mg to about 25 mg, and increased if necessary up to about 200 mg to about 1000 mg per day as either a single dose or divided doses, depending on the patient's global response. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response. In certain embodiments, a compound or composition of the invention is administered in an amount of about 1 mg/day to about 500 mg/day of the active peptide, based upon anhydrous weight of the active peptide. In some embodiments, it is administered in an amount of about 1 mg/day to about 400 mg/day of the active peptide. In some embodiments, it is administered in an amount of about 1 mg/day to about 300 mg/day of the active peptide. In some embodiments, it is administered in an amount of about 1 mg/day to about 200 mg/day of the active peptide. In some embodiments, it is administered in an amount of about 1 mg/day to about 100 mg/day of the active peptide.

A compound or composition of the invention can be administered as a single once-a-day dose or preferably as divided doses throughout a day. In some embodiments, the daily dose is administered twice daily in equally divided doses. In other embodiments, the daily dose is administered three times per day. In particular embodiments, the daily dose is administered three times per day in equally divided doses. In some embodiments, the daily dose is administered three times per day in three divided doses and each dose comprises the active peptide in an amount between about 1-100 mg, about 4-60 mg, about 4-40 mg, about 4-30 mg, about 4-25 mg, or about 4-20 mg. Preferably, the three divided doses of the composition are given around three meal times each day.

A compound or composition of the invention can be administered at various times. In some embodiments, it is administered to an enterostatin-deficient subject when the subject is fasted. In some embodiments, it is administered prior to a meal. In some embodiments, it is administered during a meal. In some embodiments, it is administered after a meal.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the composition of the invention are also encompassed by the above described dosage amounts and dose frequency schedules. Further, when a patient is administered multiple dosages of a composition of the invention, not all of the dosages need be the same. For example, the dosage administered to the patient may be increased to improve the prophylactic or therapeutic effect of the composition or it may be decreased to reduce one or more side effects that a particular patient is experiencing.

In a specific embodiment, the dosage of the composition of the invention or a composition of the invention, based on weight of the active peptide, administered to prevent, treat, manage, or ameliorate a disorder, or one or more symptoms thereof in a patient is 0.01 mg/kg, 0.05 mg/kg, 0.10 mg/kg, 0.15 mg/kg, 0.20 mg/kg, 0.25 mg/kg, 0.5 mg/kg, 0.75 mg/kg, 1 mg/kg, 1.5 mg/kg, 2 mg/kg, 3 mg/kg, 4 mg/kg, 5 mg/kg, 10 mg/kg, or 15 mg/kg or more of a patient's body weight. In another embodiment, the dosage of the composition of the invention or a composition of the invention administered to prevent, treat, manage, or ameliorate a disorder, or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

In certain embodiments, administration of the same composition of the invention may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In certain embodiments, the composition of the invention or a composition of the invention can be administered as a single, one time dose or chronically. By chronic it is meant that the composition of the invention or a composition of the invention is practiced more than once to a given individual. For example, chronic administration can be multiple doses of a pharmaceutical composition administered to a subject, on a daily basis, twice daily basis, or more or less frequently, as will be apparent to those of skill in the art. Chronic administration can continue for days, weeks, months or years if appropriate according to the judgment of the practitioner of skill.

In another embodiment, the composition of the invention or a composition of the invention is administered acutely. By acute it is meant that the composition of the invention or a composition of the invention is administered in a time period close to or contemporaneous with the onset of an event. For example, acute administration can be a single dose or multiple doses of a pharmaceutical composition administered around the onset of a meal. In some embodiments, the meal is a high calorie or high fat meal. Acute administration can also be a single dose or multiple doses of a pharmaceutical composition administered around the onset of a craving for food, specifically a craving for fatty food. A time period close to or contemporaneous with the onset of an event will vary according to the event but can be, for example, within about 30 minutes of a meal or a craving for food. In certain embodiments, acute administration is administration within about an hour of a meal or a craving for food. In certain embodiments, acute administration is administration within about 2 hours, about 6 hours, about 10 hours, about 12 hours, about 15 hours or about 24 hours after a meal or a craving for food.

In a specific embodiment, the invention provides a method of preventing, treating, managing, or ameliorating a disorder, or one or more symptoms thereof, said methods comprising administering to a subject in need thereof a dose of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compositions of the invention once every 3 days, preferably, once every 4 days, once every 5 days, once every 6 days, once every 7 days, once every 8 days, once every 10 days, once every two weeks, once every three weeks, or once a month.

The following synthetic and biological examples are offered to illustrate this invention and are not to be construed in any way as limiting the scope of this invention.

### 5. EXAMPLES

### 5.1 Example 1

### Enterostatin

For the examples below, enterostatin is obtained from commercial sources or prepared according to techniques known to those of skill in the art (see, e.g., U.S. Pat. No. 5,494,894, the contents of which are hereby incorporated by reference in their entirety.)

### 5.2 Example 2

### Pharmaceutical Compositions Comprising a Non-hygroscopic Additive

The instant example provides the following non-hygroscopic compositions comprising enterostatin.

Composition 201 4.0 mg enterostatin, 71.0 mg starch, 25 mg microcrystalline cellulose.

Composition 202 10.0 mg enterostatin, 65.0 mg starch, 25 mg microcrystalline cellulose.

Composition 203 20.0 mg enterostatin, 55.0 mg starch, 25 mg microcrystalline cellulose.

Composition 204 40.0 mg enterostatin, 35.0 mg starch, 25 mg microcrystalline cellulose.

Composition 205 60.0 mg enterostatin, 15.0 mg starch, 25 mg microcrystalline cellulose.

Composition 206 4.0 mg enterostatin, 71.0 mg starch, 25 mg dibasic calcium phosphate anhydrous.

Composition 207 10.0 mg enterostatin, 65.0 mg starch, 25 mg dibasic calcium phosphate anhydrous.

Composition 208 20.0 mg enterostatin, 55.0 mg starch, 25 mg dibasic calcium phosphate anhydrous.

Composition 209 40.0 mg enterostatin, 35.0 mg starch, 25 mg dibasic calcium phosphate anhydrous.

Composition 210 60.0 mg enterostatin, 15.0 mg starch, 25 mg dibasic calcium phosphate anhydrous.

Composition 211 4.0 mg enterostatin, 71.0 mg starch, 25 mg calcium sulfate.

Composition 212 10.0 mg enterostatin, 65.0 mg starch, 25 mg calcium sulfate.

Composition 213 20.0 mg enterostatin, 55.0 mg starch, 25 mg calcium sulfate.

Composition 214 40.0 mg enterostatin, 35.0 mg starch, 25 mg calcium silicate.

Composition 215 60.0 mg enterostatin, 15.0 mg starch, 25 mg calcium silicate.

Composition 216 4.0 mg enterostatin, 71.0 mg starch, 25 mg powdered cellulose.

Composition 217 10.0 mg enterostatin, 65.0 mg starch, 25 mg powdered cellulose.

Composition 218 20.0 mg enterostatin, 55.0 mg starch, 25 mg powdered cellulose.

Composition 219 40.0 mg enterostatin, 35.0 mg starch, 25 mg powdered cellulose.

Composition 220 60.0 mg enterostatin, 15.0 mg starch, 25 mg powdered cellulose.

Composition 221 4.0 mg enterostatin, 71.0 mg starch, 25 mg dextrose.

Composition 222 10.0 mg enterostatin, 65.0 mg starch, 25 mg dextrose.

Composition 223 20.0 mg enterostatin, 55.0 mg starch, 25 mg dextrose.

Composition 224 40.0 mg enterostatin, 35.0 mg starch, 25 mg dextrose.

Composition 225 60.0 mg enterostatin, 15.0 mg starch, 25 mg dextrose.

Composition 226 4.0 mg enterostatin, 71.0 mg starch, 25 mg lactitol.

Composition 227 10.0 mg enterostatin, 65.0 mg starch, 25 mg lactitol.

Composition 228 20.0 mg enterostatin, 55.0 mg starch, 25 mg lactitol.

Composition 229 40.0 mg enterostatin, 35.0 mg starch, 25 mg lactitol.

Composition 230 60.0 mg enterostatin, 15.0 mg starch, 25 mg lactitol.

Composition 231 4.0 mg enterostatin, 71.0 mg starch, 25 mg mannitol.

Composition 233 10.0 mg enterostatin, 65.0 mg starch, 25 mg mannitol.

Composition 232 20.0 mg enterostatin, 55.0 mg starch, 25 mg mannitol.

Composition 232 40.0 mg enterostatin, 35.0 mg starch, 25 mg mannitol.

Composition 232 60.0 mg enterostatin, 15.0 mg starch, 25 mg mannitol.

### 5.3 Example 3

### Encapsulated Compositions of Enterostatin

The present example provides non-hygroscopic encapsulated compositions of enterostatin according to the invention.

Fill 301 (% weight): 2.5% enterostatin, 42% Cremphor EL, 20% Labrasol 30% Labrafil M2125CS. Shell 301 (dry): 54% Gelatin, 18% Glycerin, 22% anidrisorb 35/70, 6% water.

The dry gelatin shell (capsule) is produced from a fluid gelatin composition using the following constituents: 42% gelatin, 10% glycerol, 10% anidrisorb, 36% water.

Fill 302 (% weight): 12% enterostatin, 40% Cremphor EL, 26% Labrasol, 22% Labrafil M2125CS, 2% Lutrol F68. Shell 302 (dry): 47% Gelatin, 28% Glycerin, 15% anidrisorb 35/70, 10% water.

Fill 303 (% weight): 5% enterostatin, 67% tocopheryl PEG-1000 succinate, 6% Cremphor EL, 6% Labrafil M2125CS, 3% ethanol, 14% propylene glycol. Shell 303 (dry): 51% Gelatin, 32% Glycerin, 12% anidrisorb 35/70, 5% water.

Fill 304 (% weight): 12% enterostatin, 28% tocopheryl PEG-1000 succinate, 22% Cremphor EL, 18% Labrafil M2125CS, 12% alpha-tocopherol, 8% propylene glycol. Shell 304 (dry): 51% Gelatin, 32% Glycerin, 12% anidrisorb 35/70, 5% water.

Fill 305 (% weight): 4% enterostatin, 42% Cremphor EL, 18% Labrafil M2125CS, 12% alpha-tocopherol, 8% propylene glycol. Shell 305 (dry): 51% Gelatin, 32% Glycerin, 12% anidrisorb 35/70, 5% water.

### 5.4 Example 4

### Solid Particulate Compositions of Enterostatin

Solid particulate compositions of enterostatin are prepared according to the invention. Excipients listed below are added to a water solution of enterostatin to yield a final solution that is spray dried according to standard techniques.

Solid particulate composition 401: 3 g enterostatin, 7 g microcrystalline cellulose.

Solid particulate composition 402: 3 g enterostatin, 3 g microcrystalline cellulose, 4 g hydroxypropyl methylcellose.

Solid particulate composition 403: 5 g enterostatin, 3 g microcrystalline cellulose, 2 g hydroxypropyl methylcellose.

Solid particulate composition 404: 3 g enterostatin, 6.95 g microcrystalline cellulose, 0.05 g silicon dioxide.

Solid particulate composition 405: 3 g enterostatin, 3 g hydroxypropyl methylcellose.

Solid particulate composition 406: 3 g enterostatin, 5 g polyethylene glycol 8000, 2 g hydroxypropyl methylcellose.

Solid particulate composition 407: 3 g enterostatin, 7 g lactose.

Solid particulate composition 408: 3 g enterostatin, 6 g mannitol, 1 g hydroxypropyl methylcellose.

Solid particulate composition 409: 3 g enterostatin, 4 g calcium phosphate tribasic, 3 g hydroxypropyl methylcellose.

Solid particulate composition 410: 3 g enterostatin, 3 g calcium phosphate tribasic, 2 g hydroxypropyl methylcellose.

Solid particulate composition 411: 2 g enterostatin, 4 g calcium sulfate, 3 g hydroxypropyl methylcellose.

Solid particulate composition 412: 3 g enterostatin, 4 g calcium sulfate, 0.05 g silicon dioxide.

### 5.5 Example 5

### Solid Dispersions of Enterostatin

The present example provides non-hygroscopic solid dispersions of enterostatin according to the invention.

Solid dispersions of this example are prepared by adding the active ingredient to melted PEG 8000 at about 67° C. with stirring. Microcrystalline cellulose is then added with further stirring. Incubation under reduced pressure at about 40° C. yields the solid dispersions of the invention.

Solid dispersion 501 2.5 mg enterostatin, 72.5 mg PEG 8000, 25 mg microcrystalline cellulose.

Solid dispersion 502 5.0 mg enterostatin, 70.0 mg PEG 8000, 25 mg microcrystalline cellulose.

Solid dispersion 503 10.0 mg enterostatin, 67.5 mg PEG 8000, 25 mg microcrystalline cellulose.

Solid dispersion 504 2.5 mg enterostatin, 72.5 mg PEG 8000, 25 mg dibasic calcium phosphate anhydrous.

Solid dispersion 505 5.0 mg enterostatin, 70.0 mg PEG 8000, 25 mg dibasic calcium phosphate anhydrous.

Solid dispersion 506 10.0 mg enterostatin, 67.5 mg PEG 8000, 25 mg dibasic calcium phosphate anhydrous.

Solid dispersion 507 2.5 mg enterostatin, 72.5 mg PEG 8000, 25 mg calcium sulfate.

Solid dispersion 508 5.0 mg enterostatin, 70.0 mg PEG 8000, 25 mg calcium sulfate.

Solid dispersion 509 10.0 mg enterostatin, 67.5 mg PEG 8000, 25 mg calcium sulfate.

Solid dispersion 510 2.5 mg enterostatin, 72.5 mg PEG 8000, 25 mg calcium silicate.

Solid dispersion 511 5.0 mg enterostatin, 70.0 mg PEG 8000, 25 mg calcium silicate.

Solid dispersion 512 10.0 mg enterostatin, 67.5 mg PEG 8000, 25 mg calcium silicate.

Solid dispersion 513 2.5 mg enterostatin, 72.5 mg PEG 8000, 25 mg powdered cellulose.

Solid dispersion 514 5.0 mg enterostatin, 70.0 mg PEG 8000, 25 mg powdered cellulose.

Solid dispersion 515 10.0 mg enterostatin, 67.5 mg PEG 8000, 25 mg powdered cellulose.

Solid dispersion 516 2.5 mg enterostatin, 72.5 mg PEG 8000, 25 mg dextrose.

Solid dispersion 517 5.0 mg enterostatin, 70.0 mg PEG 8000, 25 mg dextrose.

Solid dispersion 518 10.0 mg enterostatin, 67.5 mg PEG 8000, 25 mg dextrose.

Solid dispersion 519 2.5 mg enterostatin, 72.5 mg PEG 8000, 25 mg lactitol.

Solid dispersion 520 5.0 mg enterostatin, 70.0 mg PEG 8000, 25 mg lactitol.

Solid dispersion 521 10.0 mg enterostatin, 67.5 mg PEG 8000, 25 mg lactitol.

Solid dispersion 522 2.5 mg enterostatin, 72.5 mg PEG 8000, 25 mg mannitol.

Solid dispersion 523 5.0 mg enterostatin, 70.0 mg PEG 8000, 25 mg mannitol.

Solid dispersion 524 10.0 mg enterostatin, 67.5 mg PEG 8000, 25 mg mannitol.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A non-hygroscopic pharmaceutical composition comprising enterostatin, or a salt or solvate thereof, and a non-hygroscopic additive.

2. The non-hygroscopic pharmaceutical composition of claim 1 wherein said enterostatin is a peptide having an amino acid selected from the group consisting of APGPR (SEQ ID NO:1), VPDPR (SEQ ID NO:2) and VPGPR (SEQ ID NO:3).

3. The non-hygroscopic pharmaceutical composition of claim 1 wherein said enterostatin is a peptide having amino acid sequence APGPR (SEQ ID NO:1).

4. The non-hygroscopic pharmaceutical composition of claim 1 wherein said enterostatin is a peptide having amino acid sequence VPDPR (SEQ ID NO:2).

5. The non-hygroscopic pharmaceutical composition of claim 1 wherein said enterostatin is a peptide having amino acid sequence VPGPR (SEQ ID NO:3).

6. The non-hygroscopic pharmaceutical composition form of claim 1 wherein said non-hygroscopic additive is selected from the group consisting of dibasic calcium phosphate anhydrous, calcium sulfate, calcium silicate, powdered cellulose, dextrose, lactitol, mannitol and a mixture thereof.

7. The non-hygroscopic pharmaceutical composition of claim 1 comprising a solvate of the enterostatin.

8. The non-hygroscopic pharmaceutical composition of claim 1 comprising a hydrate of the enterostatin.

9. The non-hygroscopic pharmaceutical composition of claim 1 comprising an enterostatin salt.

10. The non-hygroscopic pharmaceutical composition of claim 9 wherein said enterostatin salt is selected from the group consisting of enterostatin chloride, enterostatin acetate, enterostatin sulfate and enterostatin phosphate.

11. The non-hygroscopic pharmaceutical composition of claim 9 wherein said enterostatin salt is enterostatin chloride.

12. The non-hygroscopic pharmaceutical composition of claim 9 wherein said enterostatin salt is enterostatin acetate.

13. The non-hygroscopic pharmaceutical composition of claim 9 wherein said enterostatin salt is enterostatin sulfate.

14. The non-hygroscopic pharmaceutical composition of claim 9 wherein said enterostatin salt is enterostatin phosphate.

15. The non-hygroscopic pharmaceutical composition of claim 1 that adsorbs less than 30% water, by weight, from 5 to 95% relative humidity.

16. The non-hygroscopic pharmaceutical composition of claim 1 that desorbs less than 30% water, by weight, from 95 to 5% relative humidity.

17. The non-hygroscopic pharmaceutical composition of claim 1 that adsorbs less than 30% water, by weight, from 5 to 95% relative humidity and that desorbs less than 30% water, by weight, from 95 to 5% relative humidity.

18. A non-hygroscopic pharmaceutical composition comprising an enterostatin within a non-hygroscopic shell.

19. The non-hygroscopic pharmaceutical composition of claim 18 wherein said shell is capable of releasing said enterostatin when administered to a subject.

20. The non-hygroscopic pharmaceutical composition of claim 18 wherein said enterostatin is a peptide having an amino acid selected from the group consisting of APGPR (SEQ ID NO:1), VPDPR (SEQ ID NO:2) and VPGPR (SEQ ID NO:3).

21. The non-hygroscopic pharmaceutical composition of claim 18 wherein said non-hygroscopic shell comprises a matrix forming material selected from non-hygroscopic matrix is selected from the group consisting of gelatins, such as type A gelatins and type B gelatins, celluloses, such as hydroxypropyl methylcellulose, starches and gum acacia.

22. A non-hygroscopic pharmaceutical composition comprising a non-hygroscopic solid dispersion of enterostatin, or a salt thereof.

23. The non-hygroscopic pharmaceutical composition of claim 22 wherein said enterostatin is a peptide having an amino acid selected from the group consisting of APGPR (SEQ ID NO:1), VPDPR (SEQ ID NO:2) and VPGPR (SEQ ID NO:3).

24. The non-hygroscopic pharmaceutical composition of claim 22 wherein said solid dispersion comprises hydroxyethylcellulose, HPC, HPMC, HPMC phthalate, PVP, PEG, polyglycolized glycerides, cyclodextrins and carbomers.

25. The non-hygroscopic pharmaceutical composition of claim 22 comprising an enterostatin salt.

26. The non-hygroscopic pharmaceutical composition of claim 25 wherein said enterostatin salt is selected from the group consisting of enterostatin chloride, enterostatin acetate, enterostatin sulfate and enterostatin phosphate.

27. A method of treating or preventing a condition related to enterostatin deficiency, comprising the step of administering to a subject in need thereof an effective amount of a pharmaceutical composition according to claim 1, 18 or 22.

28. The method of claim 27 wherein said condition is selected from the group consisting of overweight, obesity, hypertension, dyslipidemia, type 2 diabetes, coronary heart disease, stroke, gallbladder disease, osteoarthritis, sleep apnea and respiratory problems and cancer.

29. The method of claim 28 wherein said condition is obesity.

30. A method of suppressing appetite for fat in a subject in need thereof, comprising the step of administering to the subject an effective amount of a pharmaceutical composition according to claim 1, 18 or 22.


SEQUENCE LISTING (3 sequences)

SEQ ID NO: 1 - protein, 5 aa, Homo sapiens
APGPR

SEQ ID NO: 2 - protein, 5 aa, Rat
VPDPR

SEQ ID NO: 3 - protein, 5 aa, Rat
VPGPR